(19) United States
(12) Patent Application Publication
(10) Pub. No.: US 2023/0372005 A1
(43) Pub. Date: Nov. 23, 2023

(54) CARDIAC ABLATION SYSTEM AND METHOD

(71) Applicant: SUZHOU INNOVENTURES CO., LTD, Suzhou, Jiangsu (CN)

(72) Inventors: Xuwen NG, Singapore (SG); Yingxian SUN, Shenyang (CN); Tiefeng HU, Cupertino, CA (US)

(73) Assignee: SUZHOU INNOVENTURES CO., LTD, Suzhou, Jiangsu (CN)

(21) Appl. No.: 18/027,697

(22) PCT Filed: Sep. 20, 2021
(86) PCT No.: PCT/CN2021/119476

§ 371 Date: Mar. 22, 2023

(30) Foreign Application Priority Data
Sep. 22, 2020 (CN) 202011005029.0

(51) Int. Cl. A61B 18/14

(52) U.S. Cl. CPC A61B 18/1492; A61B 2018/00577

(57) ABSTRACT

A cardiac ablation system includes a catheter and a head; the head is the ablation end of the system for cardiac tissue, and the catheter is a flexible tubular connector used by the system to couple to the head. The head has a plurality of individual ablation elements; each element provided with a contact surface for contacting cardiac tissue and a flexible support body for supporting the contact surface, wherein an energy acting part is arranged on the contact surface. The ablation element has two operating states of contraction and extension at the head position of the system; in the contraction state, a plurality of individual elements aggregate with each other and present a minimum volume; in the extension state, one or more mutually individual ablation elements open and adapt to various shape changes at the point where the cardiac tissue is contacted through the contact surfaces on each element.

### FIELD OF THE INVENTION

The invention relates to the treatment of abnormal cardiac conditions such as atrial fibrillation. Specifically, the invention relates to a cardiac ablation system—a procedure to scar or destroy tissue in a heart that produces incorrect electrical signals to cause an abnormal heart rhythm.

### BACKGROUND

The treatment of abnormal cardiac conditions such as atrial fibrillation include “cardiac ablation”—a procedure to scar or destroy tissue in a heart that produces incorrect electrical signals to cause an abnormal heart rhythm.

A catheter advanced towards the heart through the patient's blood vessels, and subsequently positioned proximate to the pulmonary vein (PV) ostia. An electrical pulse is triggered and directed to the tissue through electrodes at a distal end of the catheter to electrically isolate the pulmonary veins by creating circumferential lesions. Difficulties arise as the vessel surface has a curvature the opening is a non-uniform circular opening. The current delivery systems fail to take into account the actual anatomical structure and are designed with the assumption that the vessel is a uniform tube with a uniform circular opening.

Further, the current method involving RF energy leads to tissue injury due to non-specificity and thermal energy sources. Further still, the current method is time-consuming and required a highly skilled EP to carry out the procedure

Finally, there is a risk of permanent damage if current devices are mis-positioned, therefore the accuracy of deployment is extremely important.

### SUMMARY

A cardiac ablation system for treating cardiac tissue comprises a catheter and a head; the head is the ablation end of the cardiac ablation system for cardiac tissue, and the catheter is a flexible tubular connector used by the cardiac ablation system to couple to the head. Its innovation lies in:
the head has a plurality of individual ablation elements;
each ablation element is provided with a contact surface for contacting cardiac tissue and a flexible support body for supporting the contact surface, wherein an energy acting part is arranged on the contact surface;
   The ablation element has two operating states of contraction and extension at the head position of the cardiac ablation system; In the contraction state, a plurality of individual ablation elements aggregate with each other and present a minimum volume; In the extension state, one or more mutually individual ablation elements open and adapt to various shape changes at the point where the cardiac tissue is contacted through the contact surfaces on each ablation element.

Accordingly, the invention provides an ablation head that delivers electrodes to the tissue, the head having separately operable ablation elements. Further, the ablation elements are arranged for resilient engagement so as to apply a pre-load to the tissue beyond that provided by the operator.

The energy acting part may be arranged to provide electrical impulse, RF energy or cryo-energy.

The invention provides several advantages over the prior art, including:
1. In some embodiments, the device may be able to address the unique asymmetry anatomical structure of PV ostia and vessel and accommodate both circumferentially ovoid and circular anatomical structures. Accordingly, the invention is directed to delivering energy with consideration of unique anatomy to achieve the best results.

In some embodiments, the invention may provide PEF (Pulsed electric field, abbreviated as PEF) ablation circumferentially and provide point ablation (as needed) through flexible positioning of electrodes based on the ablation needs.
3. Prior art treatment performs ablation of a lesion in a “dot-by-dot” method. The method requires EP skills to control tools precisely to ensure a continual “ablation path” is formed for a complete electrical isolated. The invention allows EP (Electro physiologist, abbreviated as EP) to carry out supported and aided isolation that is independent of EP skills. It also allows a continual lesion ablation that does not require dot-by-dot ablation.
4. Prior art treatment methods require large amounts of time for positioning and delivery or RF or other thermal ablation energy. Because of the aforementioned, the present invention may take a fraction of time for execution.

### BRIEF DESCRIPTION OF DRAWINGS

It will be convenient to further describe the present invention with respect to the accompanying drawings that illustrate possible arrangements of the invention. Other arrangements of the invention are possible and consequently, the particularity of the accompanying drawings is not to be understood as superseding the generality of the preceding description of the invention.

FIGS. 1A to 1G are various views of a first embodiment according to the present invention;

FIGS. 2A to 2C are various views of a second embodiment according to the present invention;

FIGS. 3A to 3G are various views of a third embodiment according to the present invention;

FIGS. 4A to 4G are various views of a fourth embodiment according to the present invention;

FIGS. 5A to 5H are various views of a fifth embodiment according to the present invention;

FIGS. 6A to 6E are various views of a sixth embodiment according to the present invention;

FIGS. 7A to 7F are various views of a seventh embodiment according to the present invention;

FIGS. 8A to 8B are various views of an eighth embodiment according to the present invention;

FIGS. 8C to 8D are various views of a ninth embodiment according to the present invention;

FIGS. 8E to 8F are various views of a tenth embodiment according to the present invention;

FIGS. 8G to 8H are various views of an eleventh embodiment according to the present invention;

FIGS. 81 to 8K are various views of a twelfth embodiment according to the present invention;

FIGS. 9A to 9B are various views of a thirteenth embodiment according to the present invention;

FIGS. 9C to 9D are various views of a fourteenth embodiment according to the present invention;

FIGS. 10A to 10D are various views of a fifteenth embodiment according to the present invention;

FIGS. 11A to 11H are various views of a sixteenth embodiment according to the present invention;

FIGS. 12A to 12F are various views of a seventeenth embodiment according to the present invention;

FIGS. 13A to 13B are various views of an eighteenth embodiment according to the present invention;

FIGS. 13C to 13D are various views of a nineteenth embodiment according to the present invention;

FIG. 13E is an isometric view of a twentieth embodiment according to the present invention;

FIG. 13F is an isometric view of a twenty first embodiment according to the present invention;

FIGS. 14A to 14K are various views of a twenty second embodiment according to the present invention;

FIGS. 15A to 15C are various views of a twenty third embodiment according to the present invention;

FIGS. 16A to 160 are various views of a twenty fourth embodiment according to the present invention;

FIGS. 17A to 17J are various views of a twenty fifth embodiment according to the present invention;

FIGS. 18A to 18D are various views of a twenty sixth embodiment according to the present invention;

FIGS. 19A to 19F are various views of a twenty seventh embodiment according to the present invention;

FIG. 20A is a schematic of electroporation voltage delivery (PV cross section) after application of pulse energy ablation device;

FIG. 20B is a schematic of ablation energy (PV cross section) after application of pulse energy ablation device;

FIG. 20C is a schematic of ablation pattern using traditional RF treatment (left) and ablation pattern using pulse ablation of device;

FIGS. 21A to 21G are various views of various embodiments according to the present invention;

FIGS. 22A to 22F are various flow charts of process according to various embodiments of the present invention;

### DETAILED DESCRIPTION

The invention is further described in combination with the attached drawings and embodiments below:

The invention is intended to deliver pulse energy to destroy tissue in a heart that produces incorrect electrical signals. For example, the tip of the invention is delivered via a catheter into the left atrium (LA) of the heart. Depending on anatomy specification or operator preference, the device may be deployed to the pulmonary vein (PV) ostia or into the PV via a catheter system for patients with atrial fibrillation.

The pulse energy may be in different forms including cryo-energy, radio frequency (RF) or electrical pulses, so that the energy acting part may be arranged to provide electrical pulses, radio frequency (RF) energy or cryo-energy, wherein the RF energy may be provided by the RF generator, the energy acting part may use electrodes; The pulse energy may be generated by a DC generator, and the energy acting part may use electrodes; The cryo-energy may be provided by an argon generator, and the energy acting part may be arranged in a manner similar to that of a cryo-probe. The energy acting part is positioned on the respective elements corresponding to the type of energy being utilized. It will therefore be appreciated that, while the following embodiments generally refer to transmitting an electrical pulse, these arrangements may equally be adapted for use with transmitting other forms of energy including, but not limited to, cyro-energy and RF pulse system instead.

An important feature of the present invention is its ability to provide a resilient engagement with the tissue. Essentially, the ablation elements provide an active pressure against the tissue, gaining better engagement and either molding about the tissue (resilient deformation) or positioning about the tissue (resilient displacement). In either case, the ablation elements act separately through deformation or displacement to find an equilibrium position for better contact. This is particularly advantageous when one considers the variability in the shape of the tissue, and the need to have better engagement for a more efficient treatment. Because of the resilient engagement, the operator does not need to make minor and/or repeated positional changes to obtain the best position. In finding a resilient equilibrium position, the ablation elements position about the tissue automatically.

Taking the use of electrodes in the energy acting part as an example, with reference to the following embodiments, for device deployment to the PV ostia, various embodiments of the present invention include:
1. A device with multiple electrodes (with or without force sensors);
2. A device with multiple electrodes+features to aid better electrodes alignment to vessel wall;
3. A device with multiple electrodes+features to aid better electrodes alignment to vessel wall+anchorage feature.

For device deployment into the PV vessel, various embodiments of the present invention include:
4. A device with multiple electrodes (with or without force sensors)+separate anchorage feature;
5. A device with multiple electrodes deployment simultaneously with anchorage;
6. A device with electrodes design to achieve good adherence to vessel wall—when operator manoeuvres it.

Once the tip of the device (with the electrode and/or sensors) is positioned onto the PV ostia or PV vessel, a direct pulse energy is applied via a generator. This step is repeated sequentially until the incorrect electrical signals are completely isolated.

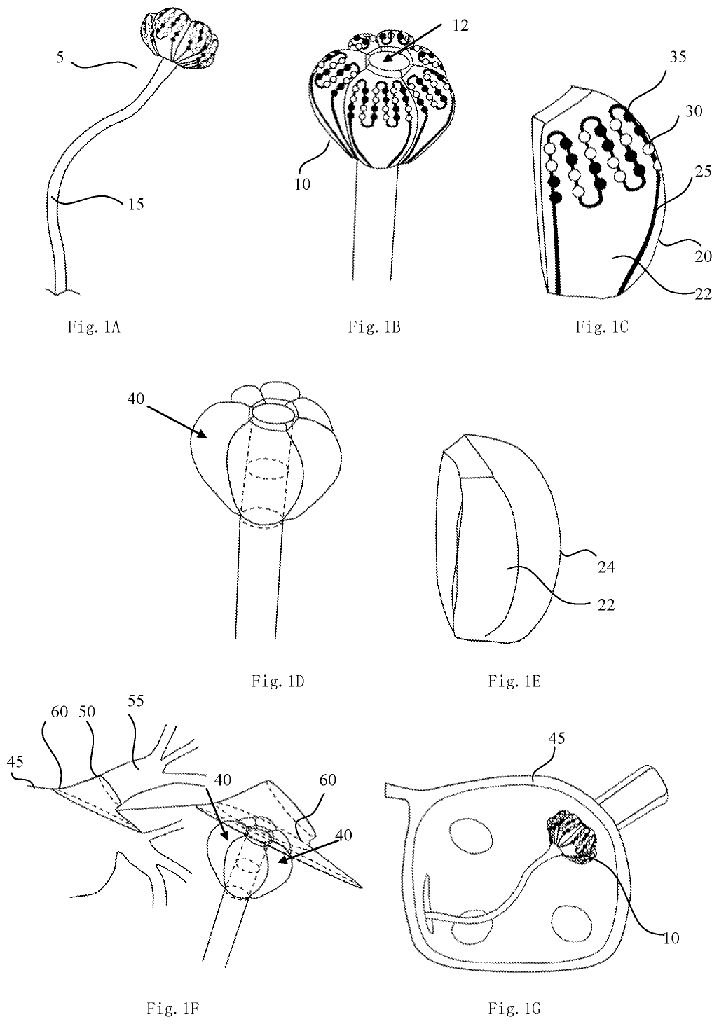

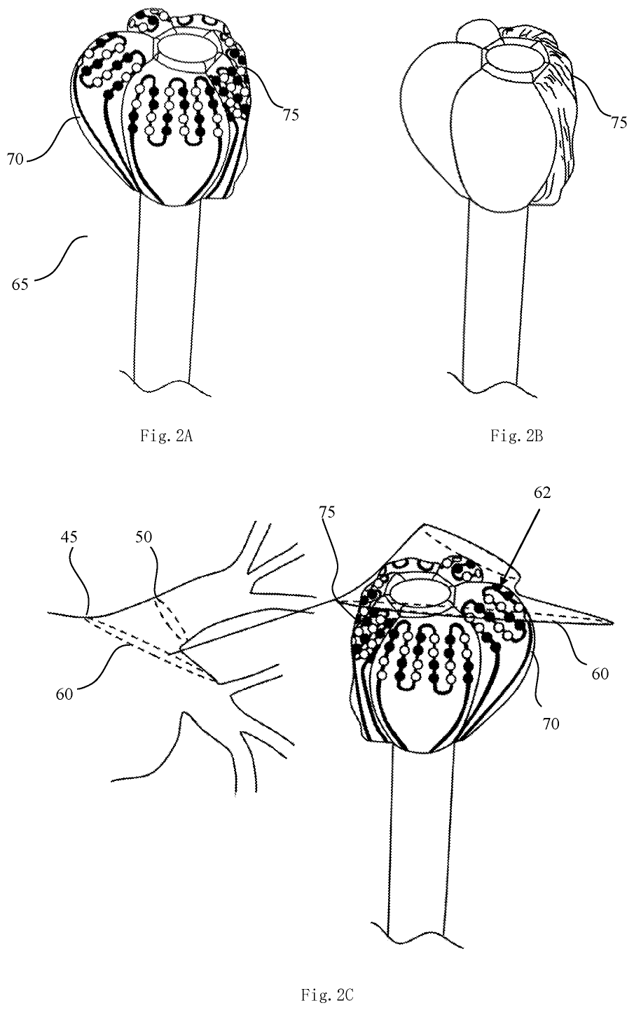

Considering specific embodiments of the present invention, and first embodiment is shown in FIGS. 1A to 1G.

Here an ablation device 5 comprises an ablation head 10 mounted to a catheter 15. The catheter 15 acts to insert the ablation head 10 into position within the heart 45, and in particular about the ostia 60 of the pulmonary vein 55. The area between cross section tissue 50 and cross section tissue 60 is a tissue treatment area.

The head 10 is inserted uninflated, until in position, whereby ablation element 20 is inflated by passing a fluid through the catheter 15, such as air (or other gas) or water (or other liquid).

It will be noted that the head 10 include a plurality of ablation elements 20, and in one of these embodiments, six ablation elements 20, which are positioned circumferentially about a centre 12 of the head 10. The ablation elements 20 include electrodes on the contact surface 22 of the ablation element 20. On inflation, the ablation element 20 provides a resilient engagement, through resilient deformation of the inflated element, with the tissue intended to be destroyed as part of the intended treatment.

Each ablation element 20 includes electrodes embedded in the contact surface 22 for passing the electrical pulse to the electrodes 30. When in contact with the tissue, the electrical pulse is directed through the electrodes according to the method described above, and also in relation to the methods defined in FIGS. 22A to 22F. In this embodiment, force sensors 35 are also included, which are arranged to feedback to the operator that contact with the tissue has been made, and the degree of resilient engagement applied to the tissue. The arrows in FIGS. 1D and 1F indicate the resilient engagement forced direction 40 of the ablation element 20, may assist in ensuring complete contact with the tissue, and so facilitate a faster and more efficient treatment. The data from the sensors may also help determine the position and proximity of the electrodes. It will be appreciated that, while optional, sensors may be included within the network for temperature monitoring, contact forces monitoring, impedance monitoring and tactile feedback.

FIGS. 2A to 2C show an alternative embodiment to that of FIGS. 1A to 1G; which is the second embodiment. Here, only some of the ablation elements 70 have been inflated, and so only the inflated side provides the resilient deformation against the tissue, the arrow in FIG. 2C indicating the resilient deformation forced direction 62 of the inflated ablation element 70. The other elements 75 remain uninflated and so providing the head 65 with an irregular shape to suit the anatomical structure irregularity of the PV ostia 60 tissue. Thus, for this embodiment, the selective inflation of the ablation elements 70, and so the ability to keep some ablation elements 75 uninflated, provides the ablation head 65 with the capacity to fit to a range of shapes and thus extending its applicability, as compared to the uniform shape adopted by prior art devices.

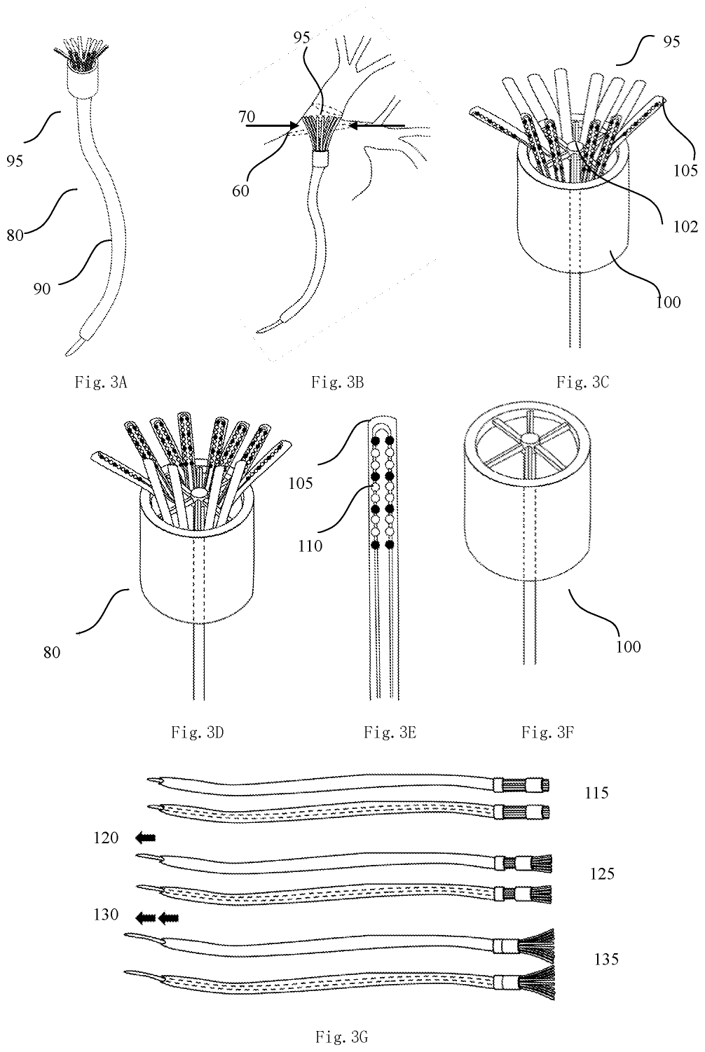

FIGS. 3A to 3G show a further device 80 according to one embodiment of the present invention, which is the third embodiment. Important departures of this device 80 from that of the first two embodiments has the inflated ablation elements replaced with relatively stiff elongate ablation elements 105. These elements are not entirely “rigid” and may have elastic properties to allow them to “bend” when force is applied. In this case, they may return to their initial shape once the force is removed. These elements could be made of elastomers/silicon resin. The head 95 of the device 80 includes a plurality of the elongate ablation elements 105, mounted to a catheter 90. The elongate ablation elements 105 are flexurally spring loaded to project radially from the centre 102 of the head and so are resiliently displaced about the tissue 60. The arrow in FIG. 3B indicates the resilient displacement forced direction 70 of the ablation element 105, and the arrow in FIG. 3G indicates the movement direction of the housing 160 when performing actions 1120 and 130.

Delivery to the heart requires restraint of the elongate members 105, and so a selectively movable housing 100 is used.
On action 120 by an operator, the housing 100 is arranged to move from a first position 115 restraining the elongate ablation elements 105 to an intermediate 125 and subsequently action 130 is performed to move the housing 100 to the proximal position 135 of the fully deployed arrangement. In this embodiment, action involves retracting the housing 100 from a distal position towards the proximate position of the catheter 90.

Once in place, electrodes 110 in the contact face of the elongate ablation elements 105 direct the electrical pulse as already described.

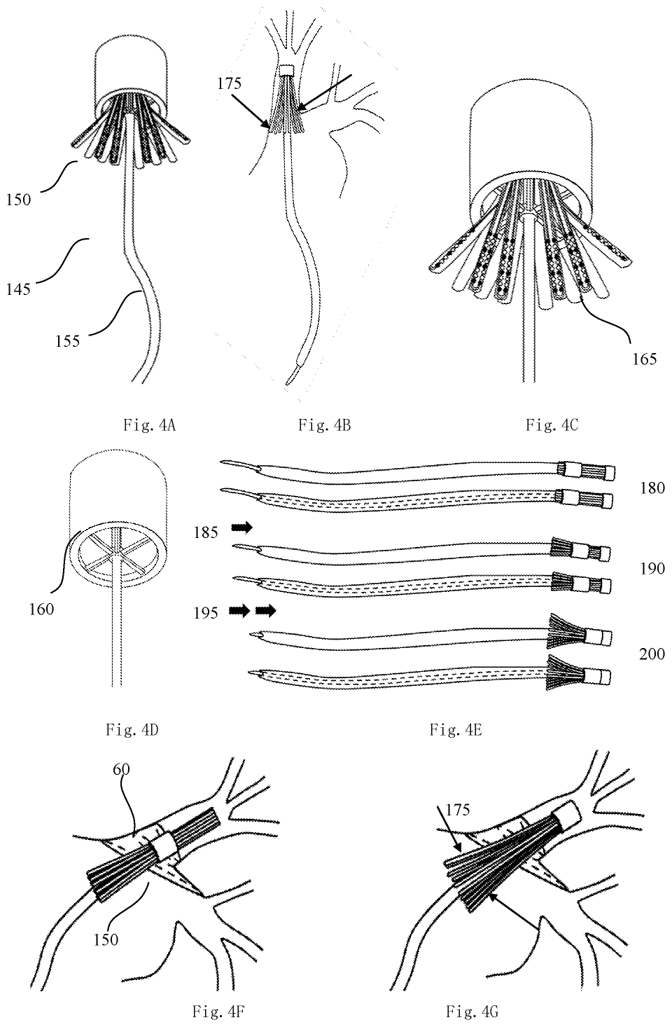

FIGS. 4A to 4G show a similar embodiment to that of FIGS. 3A to 3Q which is the fourth embodiment, in that the device 145 has a head 150 mounted to a catheter 155. The head 150 comprises elongate ablation elements 165, however rather than generally projecting forward towards a distal end of the device, in this embodiment, the elongate ablation elements 165 project towards the proximate end. When released from the housing 160 to the distal position 200, the elongate ablation elements 165 are flexurally spring loaded so as to project radially in order to be resiliently displaced about the tissue 60. However, as the elongate ablation elements 165 project in an opposite direction to that of the earlier embodiment, the housing restraining the elements moves towards a distal end of the device in order to release the elongate ablation elements 165. The operator, in this case, pushes the release mechanism forward perform actions 185 and 195 to move the housing 160. Once in the appropriate position, the housing 160 is moved to position the housing in the proximal position 180, the middle position 190, and the distal position 200 to fully deploy the head 150 into position. The arrows in FIGS. 4B and 4G indicate the resilient displacement forced direction 175 of the ablation element 165, and the arrows in FIG. 4E indicate the movement direction of the housing 160 when performing actions 185 and 195.

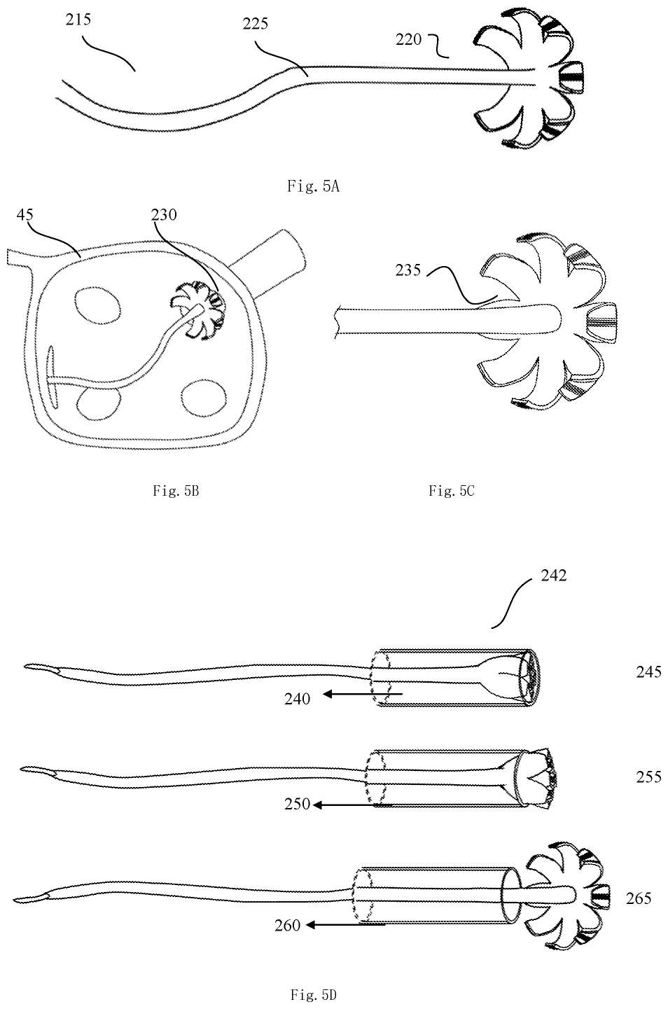

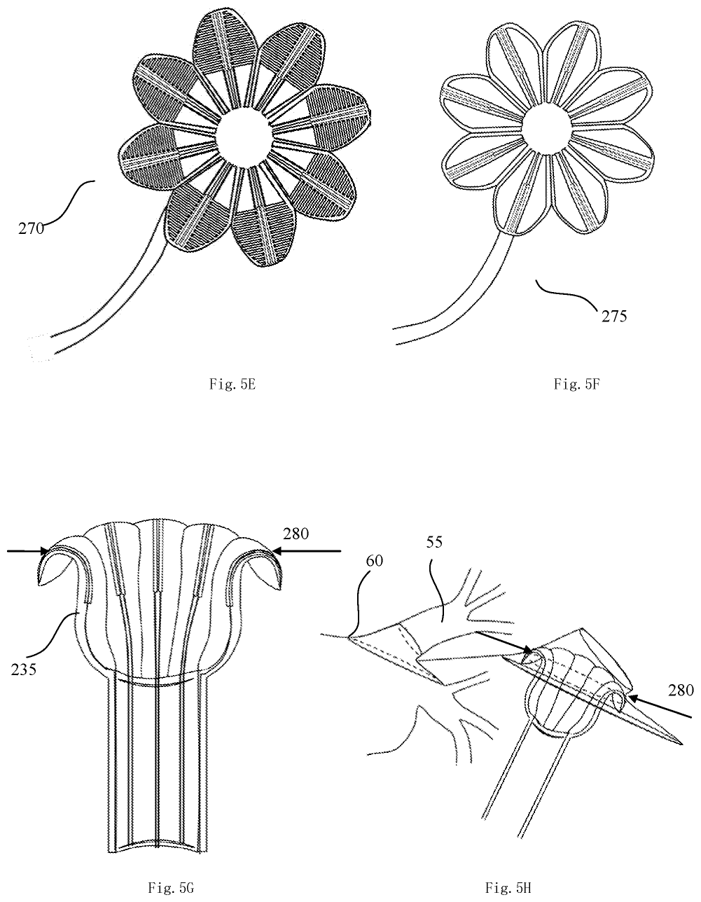

FIGS. 5A to 5H show a further embodiment, which is the fifth embodiment. Here the device 215 includes a flower shaped head 220, having petal shaped ablation elements 235 projecting radially from the centre of the head 220. The petal shaped ablation elements 235 are curved to form a flexural spring, and consequently resiliently deformed against the tissue 230 in this manner. The arrows in FIGS. 5G and 5H indicate the resilient displacement forced direction 280 of the ablation element 235.

Given the broad head, for insertion a housing 242 similar to that of FIG. 2 is used to restrain the petal shaped elements until properly positioned. Once in place, the housing 242 is shifted, and actions 240, 250, 260 are performed, gradually removing the restraint to the first position 245, the second position 255, and the third position 265 for full deployment of the head 220.

The flat shape of the petal shaped ablation elements 235 allows for various patterns of electrodes, such as the first pattern 270 and the second pattern 275. In particular, in the first pattern 270, the flat shape allows the use of strain gauges for the force sensor. In this arrangement, contact with the tissue involves bending of the ablation element 235, and so triggering a response through the strain gauge for action by the operator. The second pattern 275 shows the use of thin film force sensors as an alternative. It will be appreciated that several different methods of providing force detection are possible, with the scope of the invention not limited by any one method.

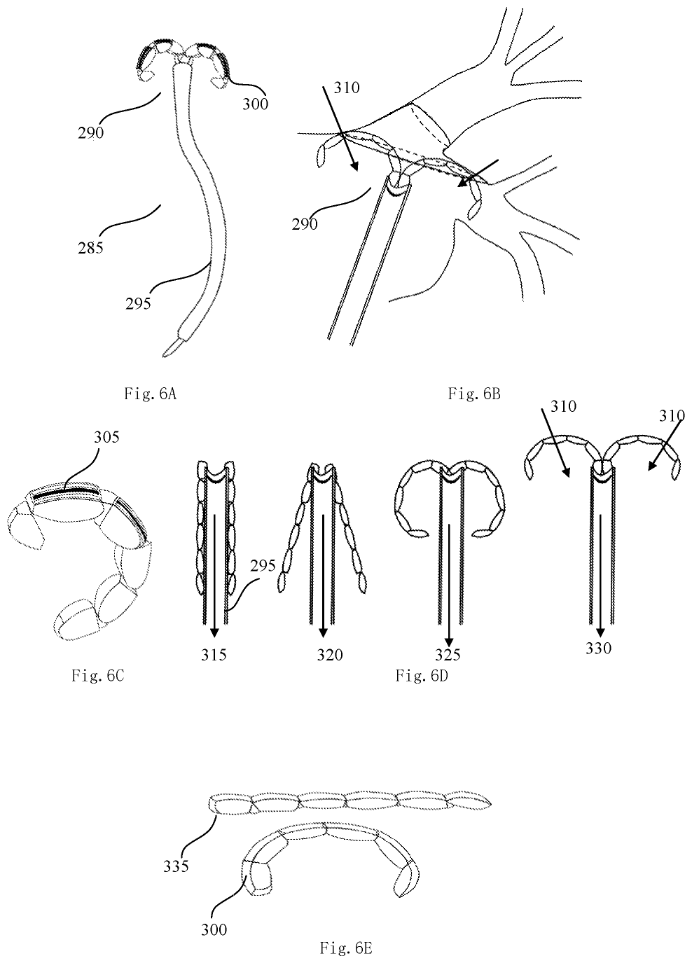

A further embodiment is shown in FIGS. 6A to 6E, which is the sixth embodiment. Here the device 285 includes a head 290 having ablation elements 300 in the form of linkages.

The ablation elements 300 includes a series of segments 335 which are pivotally connected in series allowing an articulation between the segments 335 and consequently for the ablation element 300 to flex within a plane.

On insertion, the ablation elements resides parallel with the catheter 295 on an outside surface. Once in place, the deployment is activated with a force applied to the end of the linkage coupled to the catheter 295.

As the force increases to perform actions 315, 320, 325, and 330, the linkage element is gradually expanded so as to adopt a curved arrangement similar to a circular arc of up to 180°, emulating a “scorpion tail” shape. Segments 330 along the linkage (though not necessarily all), may have electrodes 305 attached, and so the elements are capable of applying an electrical pulse along the curved surface of the linkage. The pivot connection between the segments also allows for a resilient displacement when contact is made with the tissue. The oblique arrows in FIGS. 6B and 6D indicate the resilient displacement forced direction 310 of the ablation element 165, and the vertical downward arrows in FIG. 6D indicate the action directions of actions 315, 320, 325, and 330.

Thus, the head 290 provides a very broad curved surface to contact the tissue. An advantage of this arrangement is therefore the capacity to allow for significant variability in the width of the tissue to be treated. The span covered by the ablation elements 300 is only dependent upon the number of segments 335 adding to the length of the ablation elements 300. The retracted position of the ablation elements 300 on insert, being parallel to the catheter 295 theoretically means the ablation elements 300 in the form of linkages can be extraordinarily long, and so having a considerable ablation width once deployed.

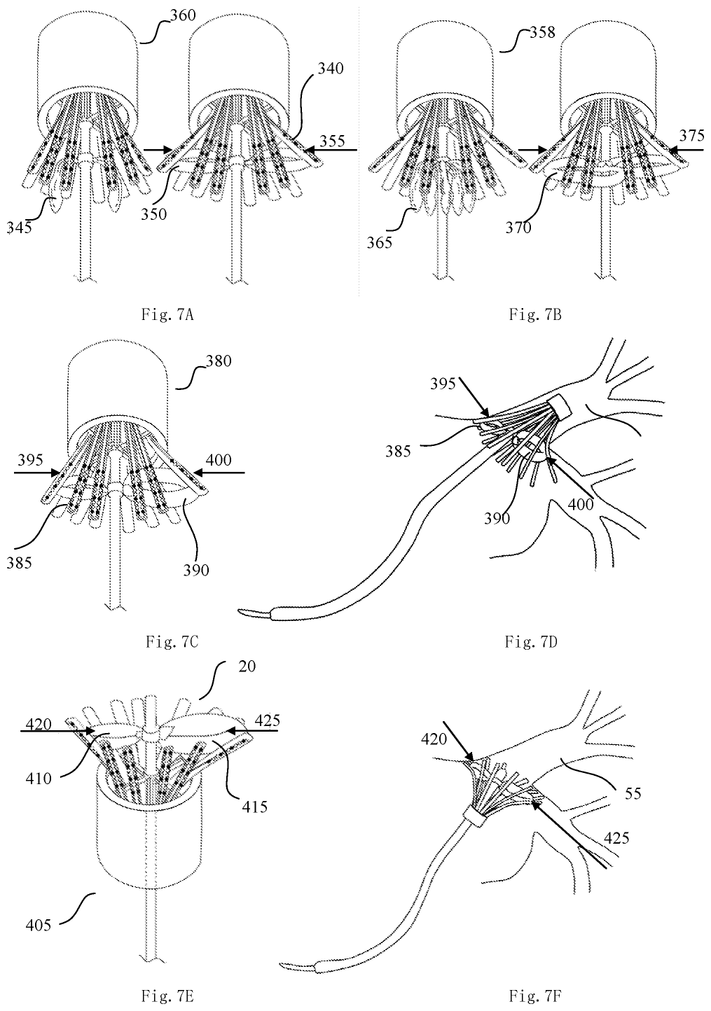

FIGS. 7A to 7F show a similar embodiment to those of FIGS. 3 and 4, which is the sixth embodiment. The respective ablation heads 360,358,380,405 are formed using the elongate ablation elements. However in these embodiments, inflatable balloons are included centrally to the elongate ablation elements. On deployment, the housing is retracted, the elongate ablation elements project outwards assisted by the inflation of the balloons 350, 370, 390 415. Thus these embodiments demonstrate both resilient forms of contact through displacement of the ablation and deformation by the balloons.

In these embodiments, the deployment is a three step process. In one step, the housing is retracted. The elongate ablation elements then radially expand and the balloons go from an uninflated condition 345, 365 to an inflated condition 350,370. The balloons may be single ring (not shown), or a plurality of balloons placed about the centre.

An interesting aspect on these embodiments is where different balloons are placed about the centre. In some cases, on inflation, the resilient engagement varies, with a smaller balloon providing a smaller expansion of the elongate ablation elements and the larger balloon a larger expansion. This, in turn, provides a differential resilient engagement with a smaller displacement 395, 420 for the smaller balloon and a larger displacement 400, 425 for the larger balloon, so as to accommodate anatomical angles of the ostia/PV. Thus, these embodiments also provide for variation in the shape and size of the tissue to be treated.

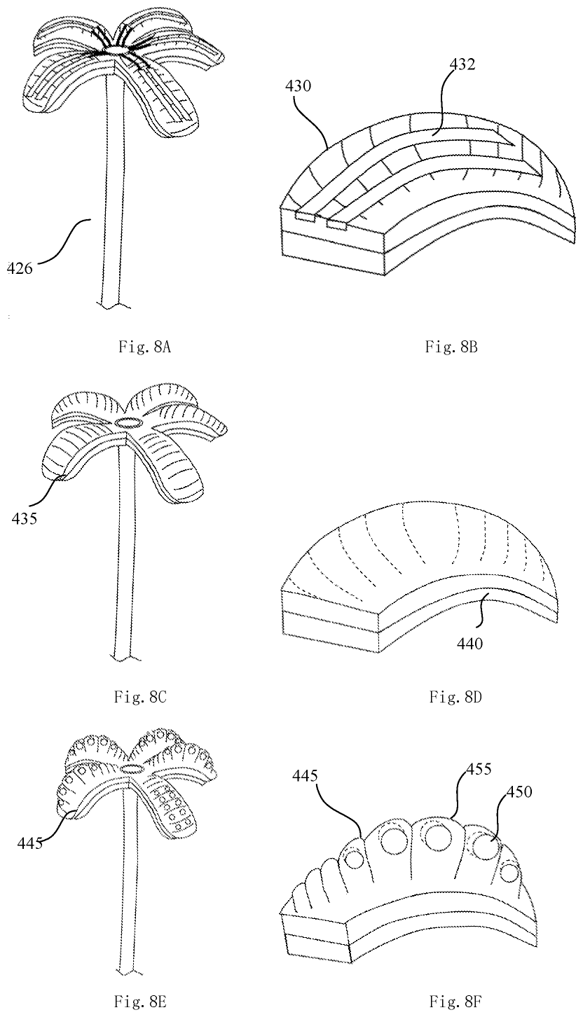

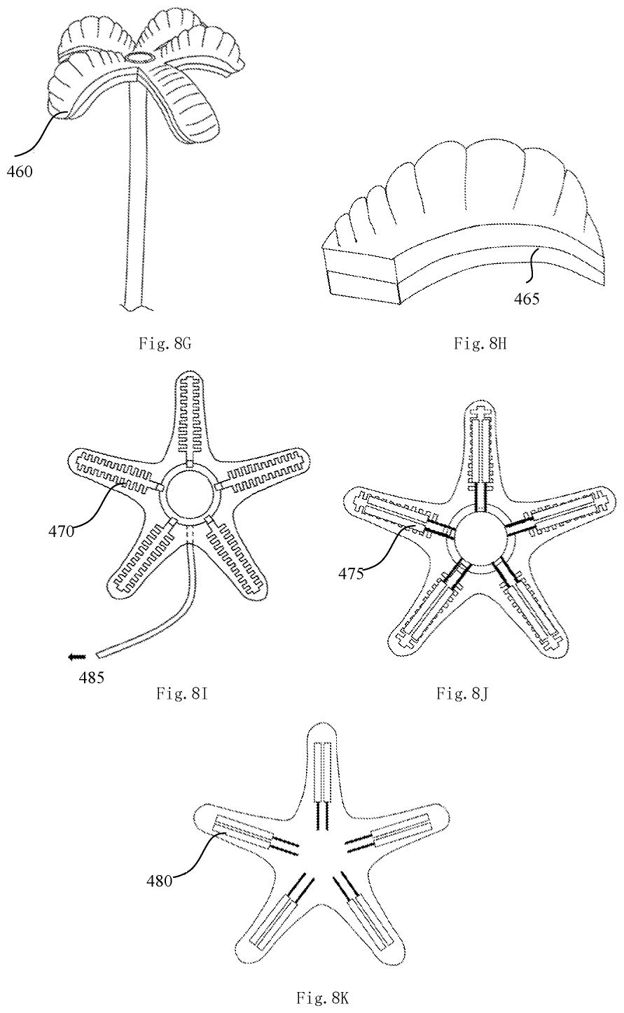

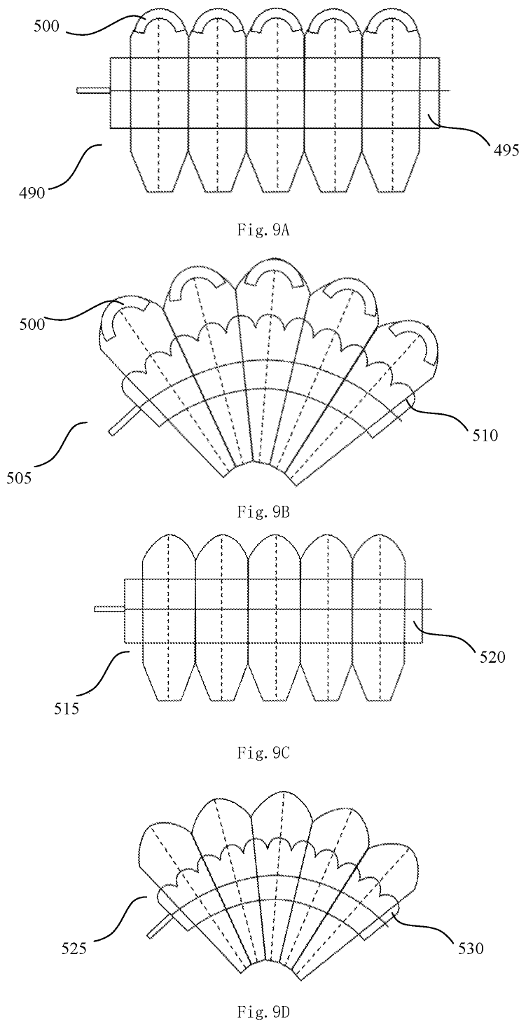

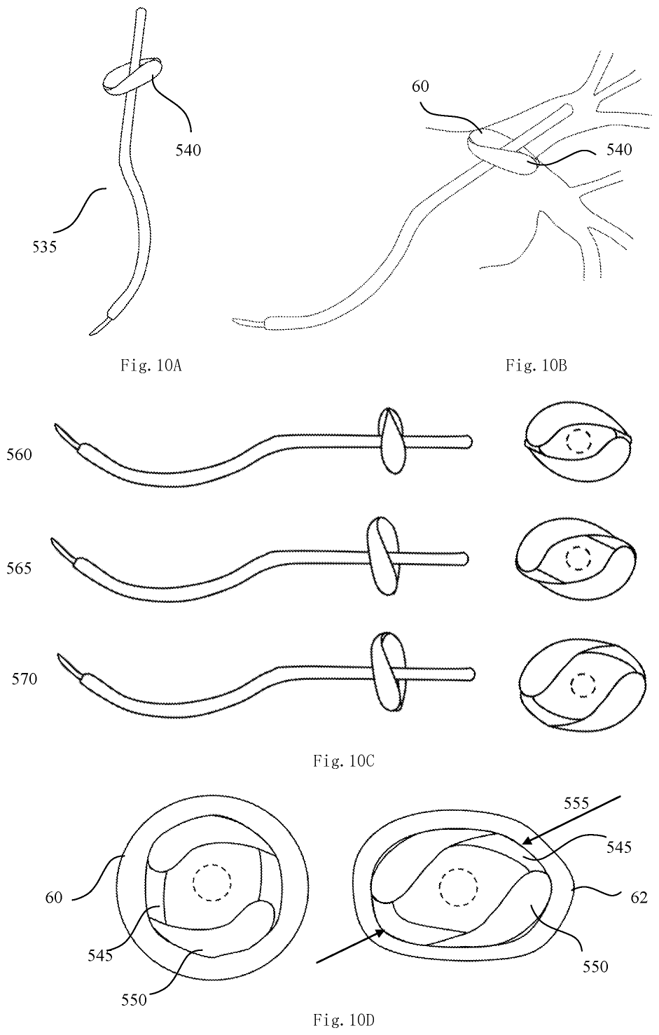

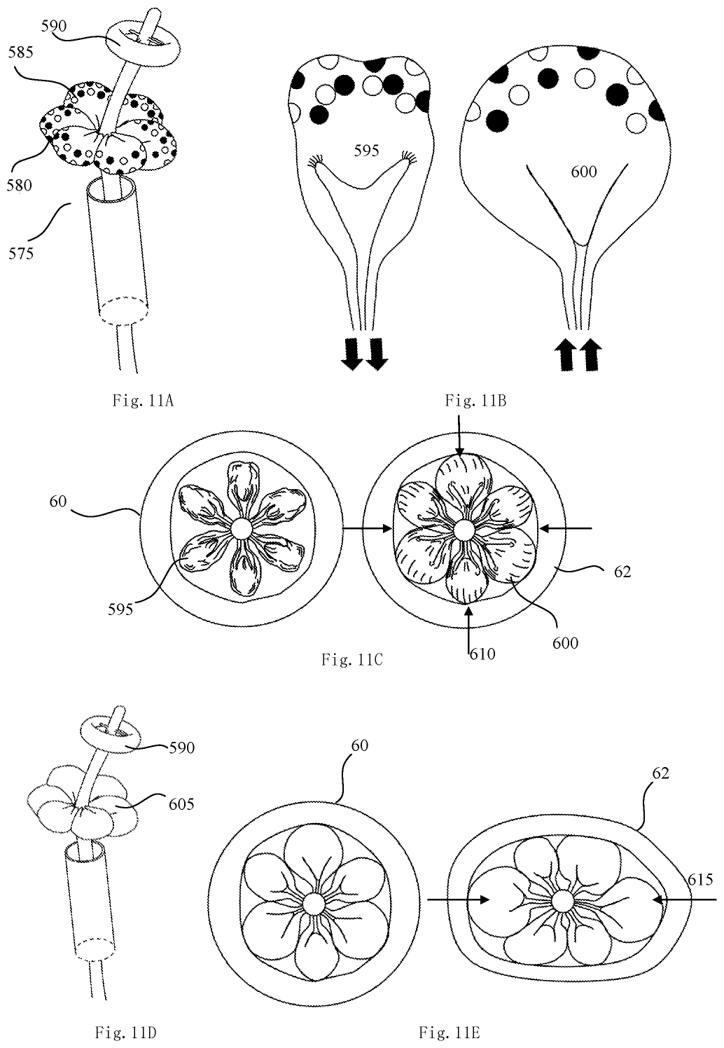

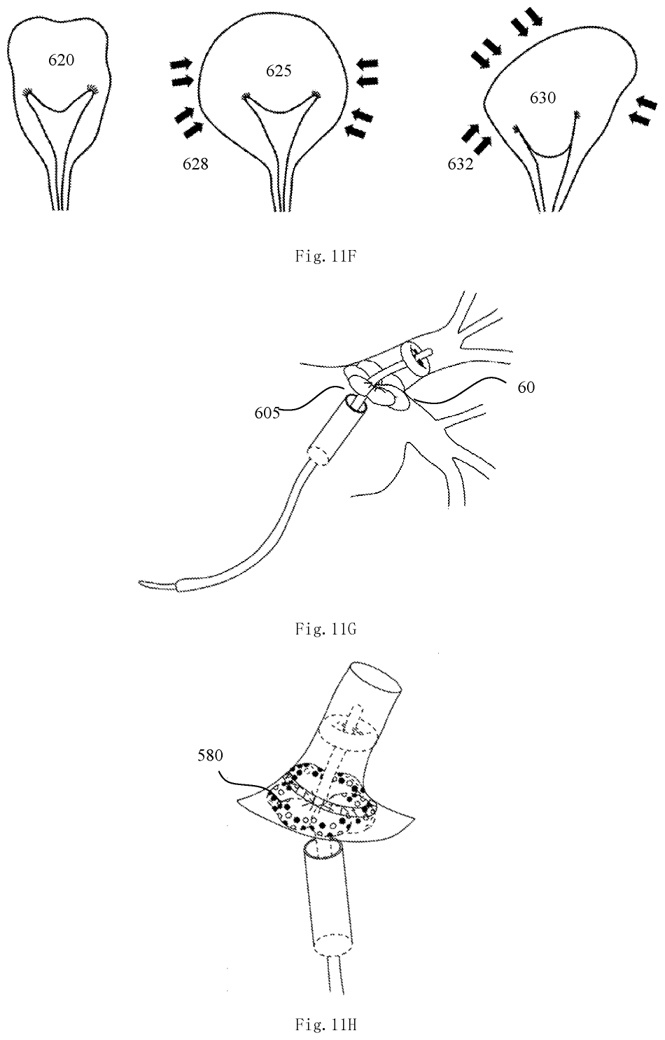

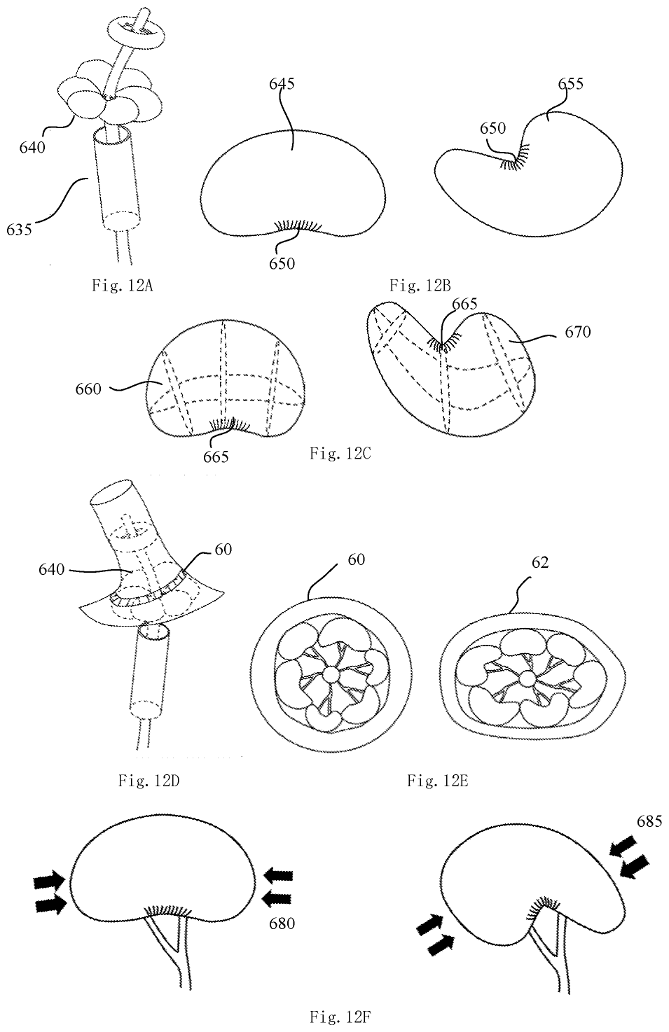

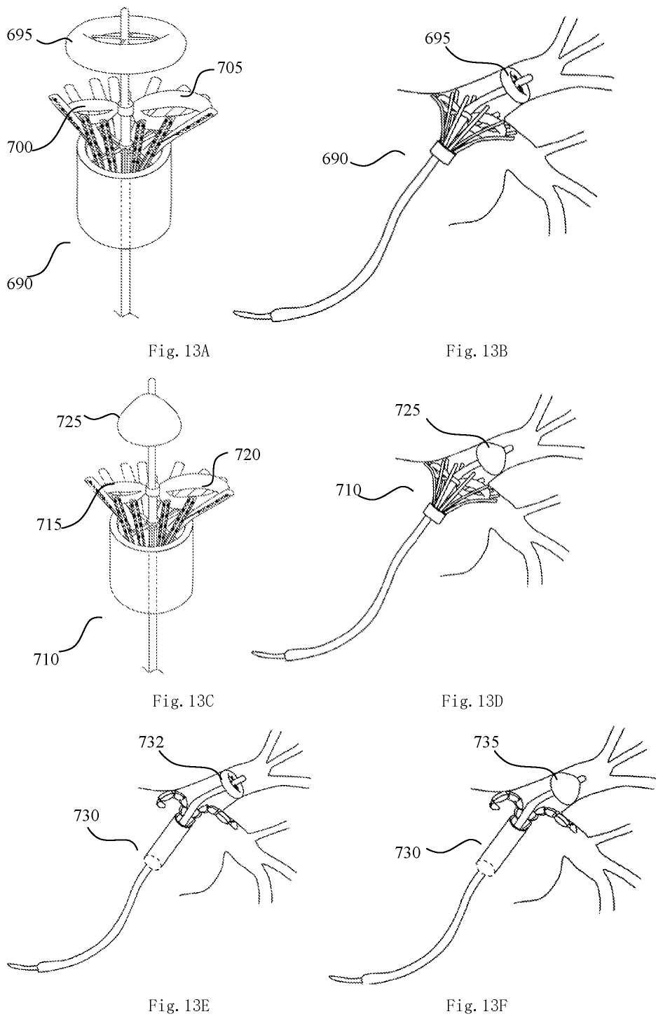

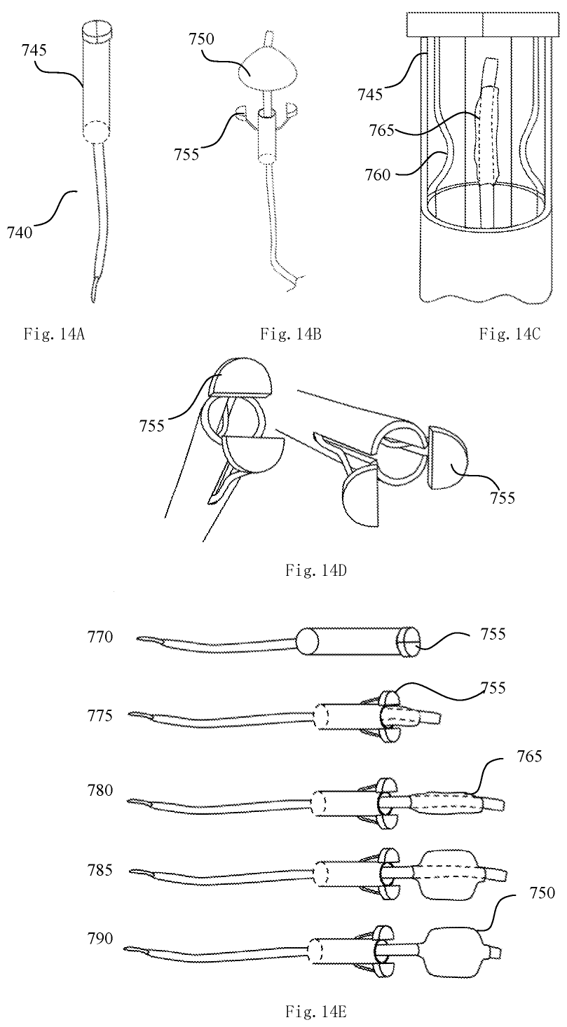

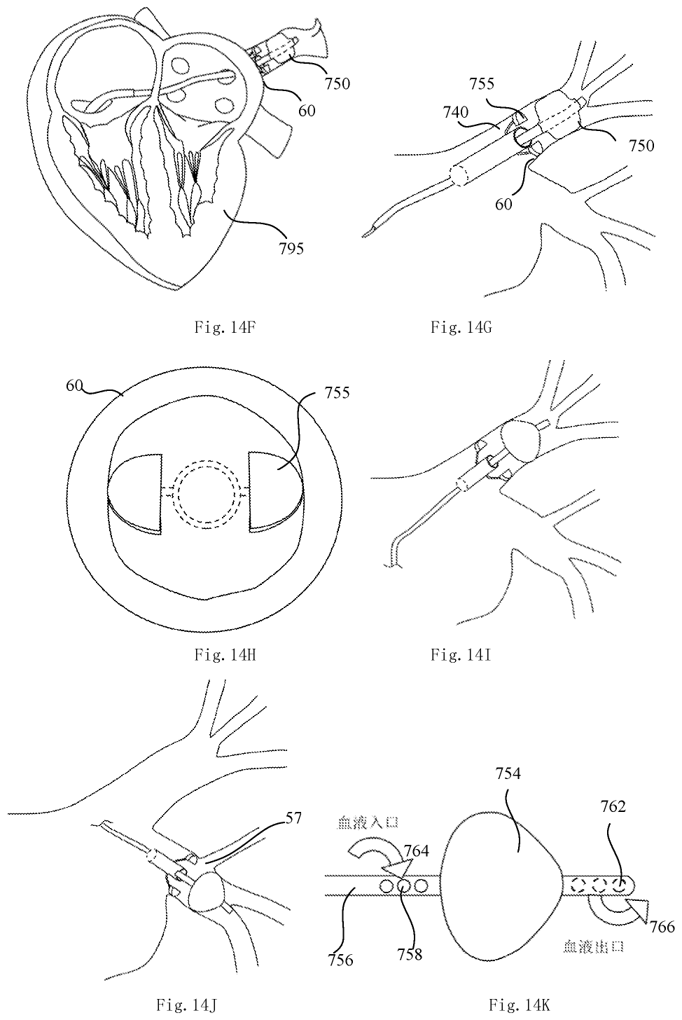

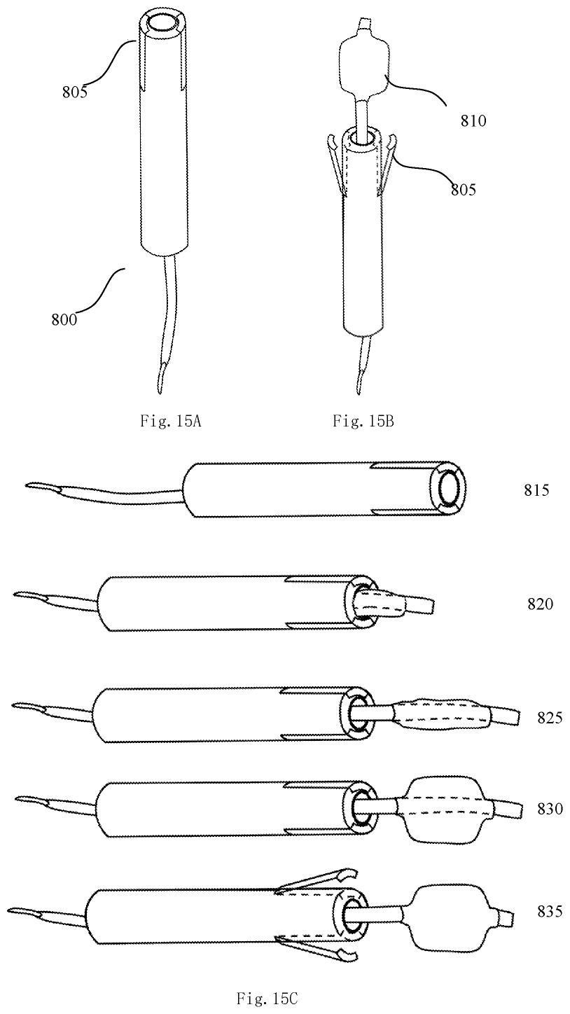

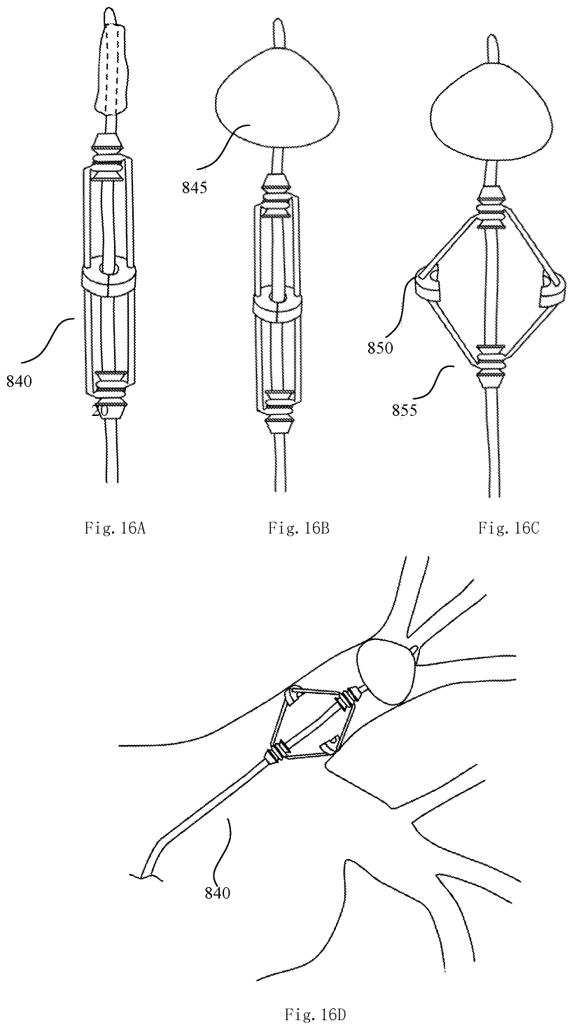

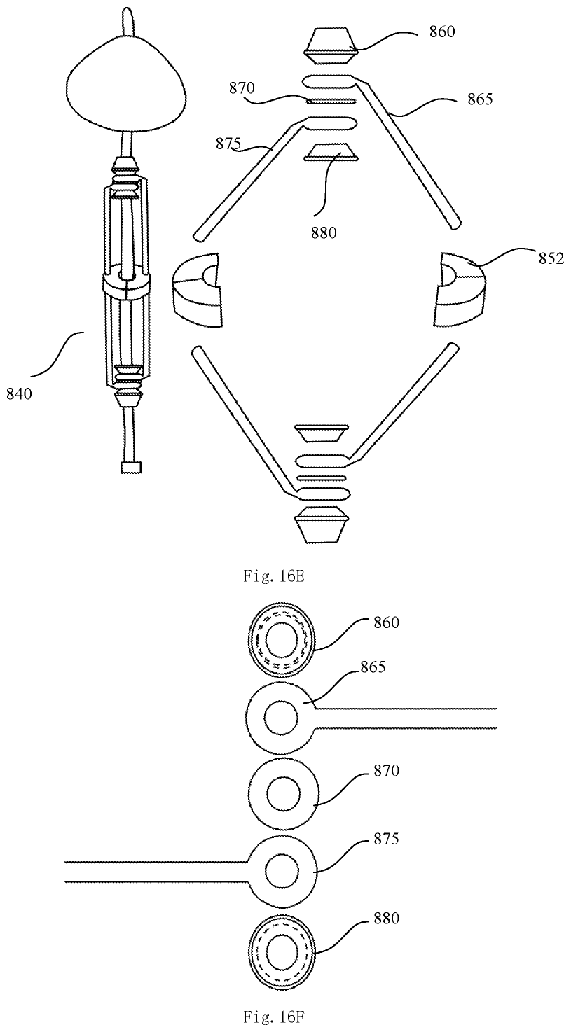

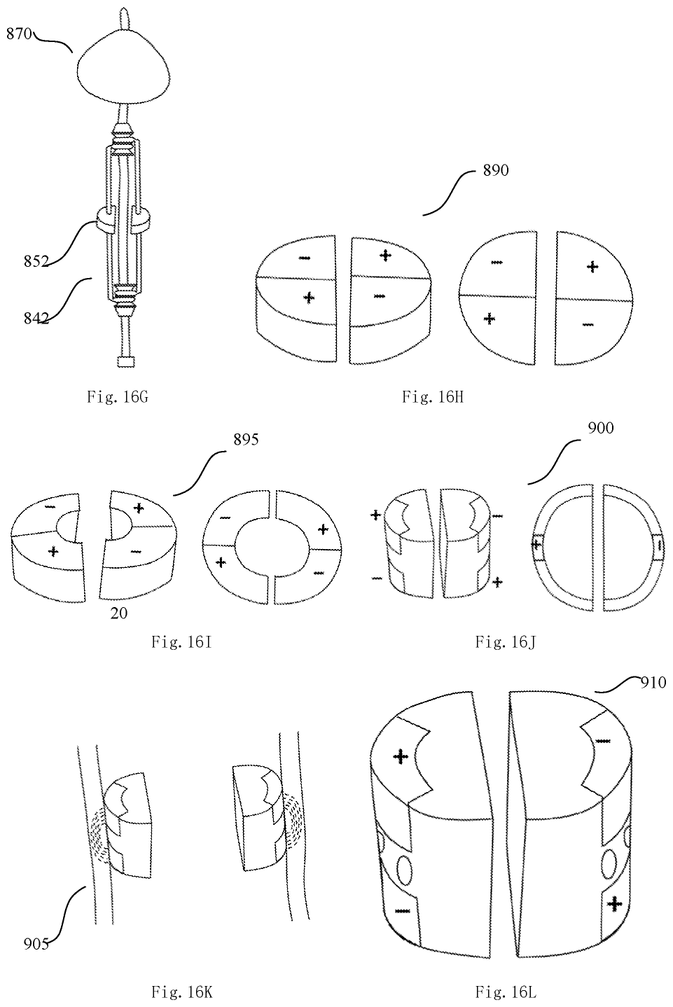

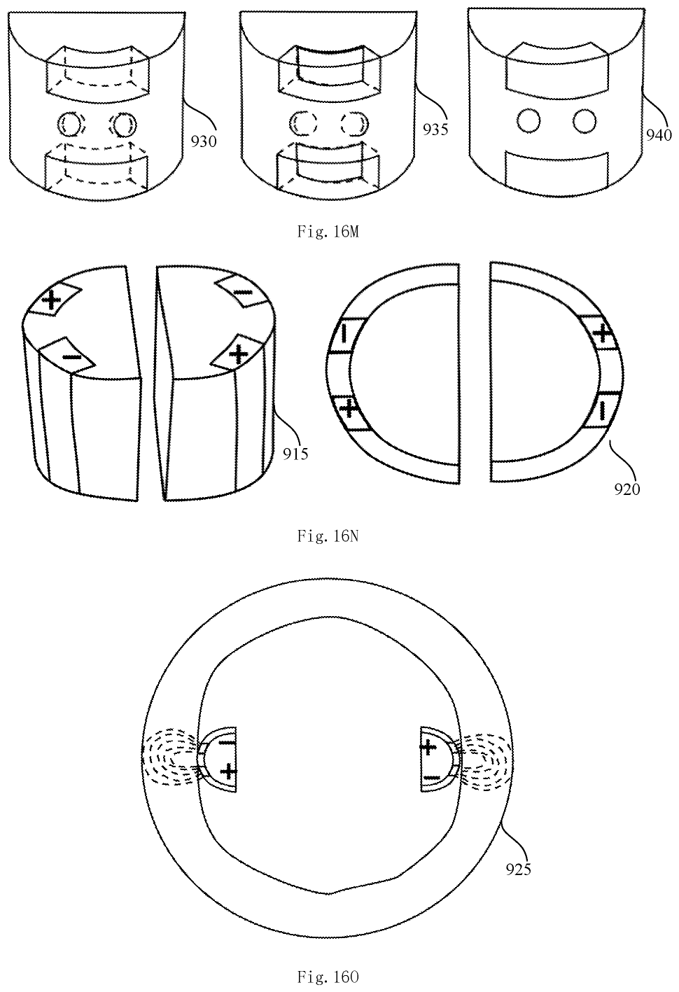

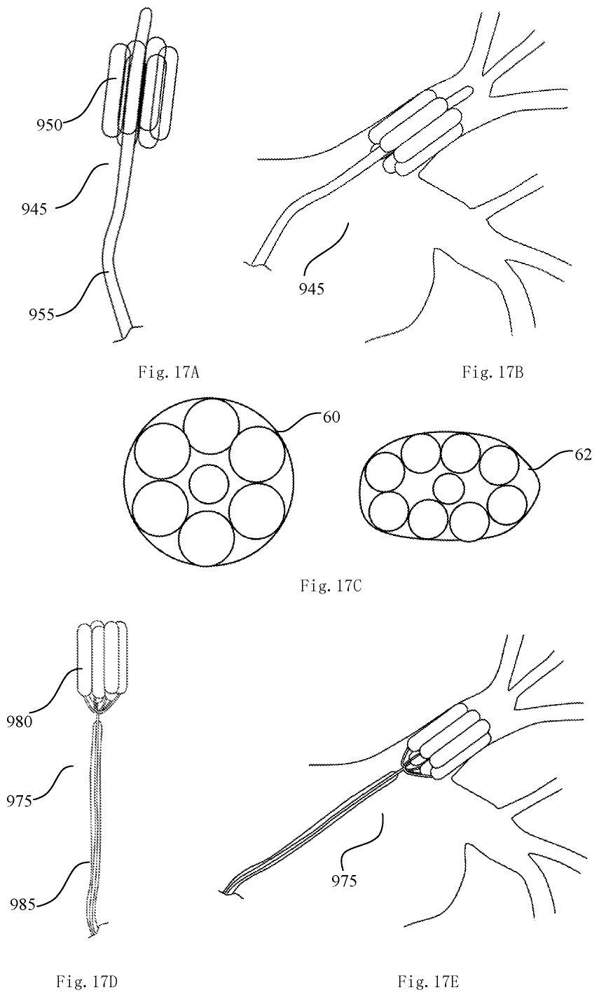

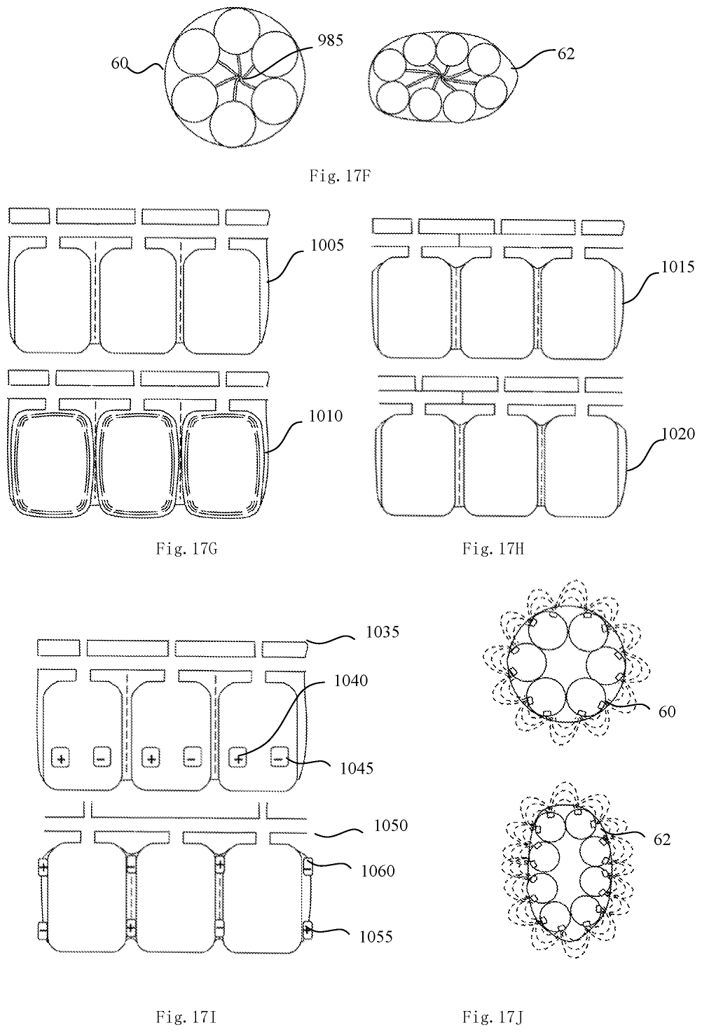

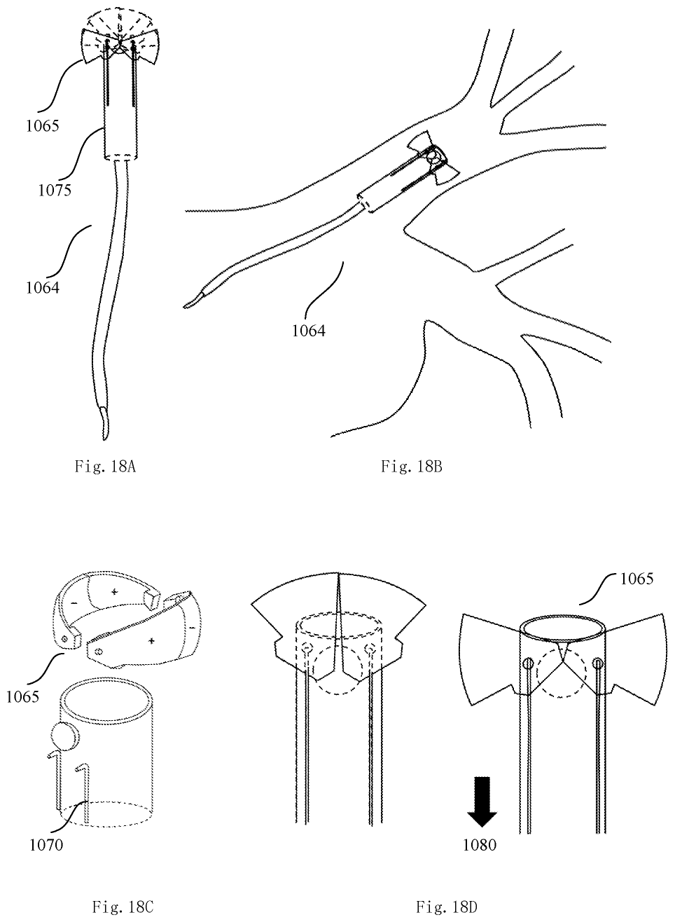

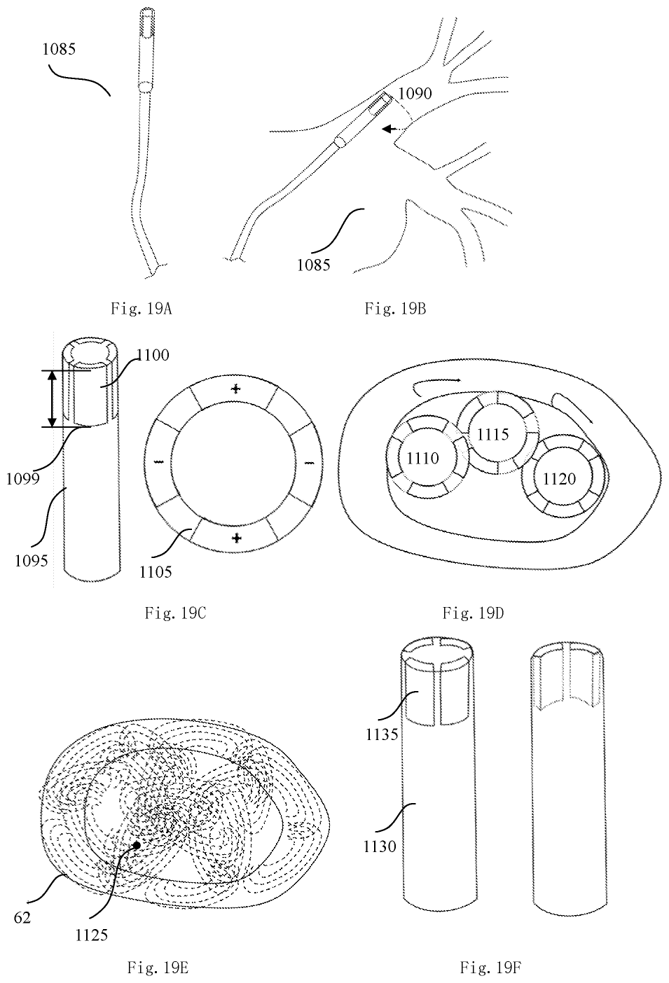

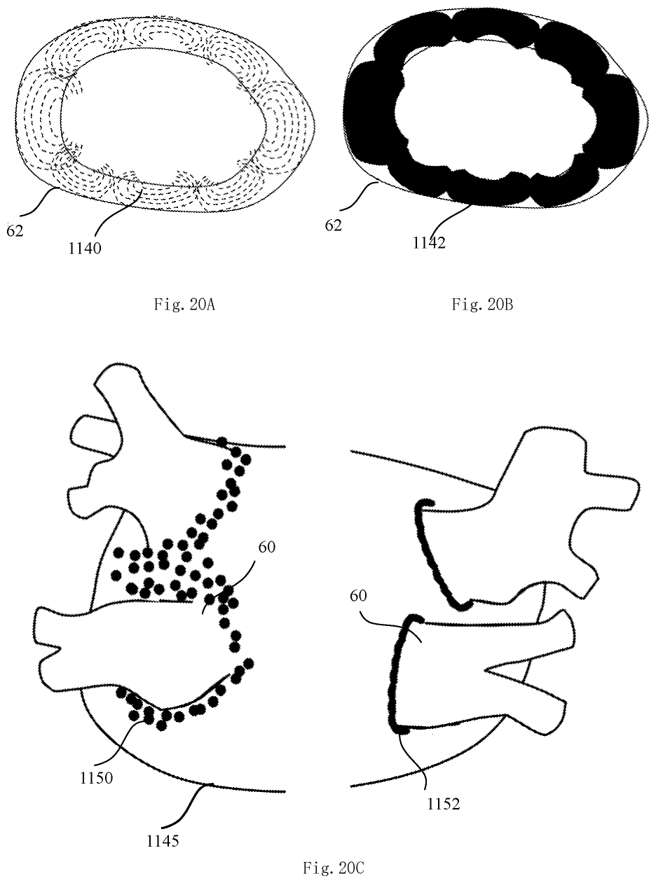

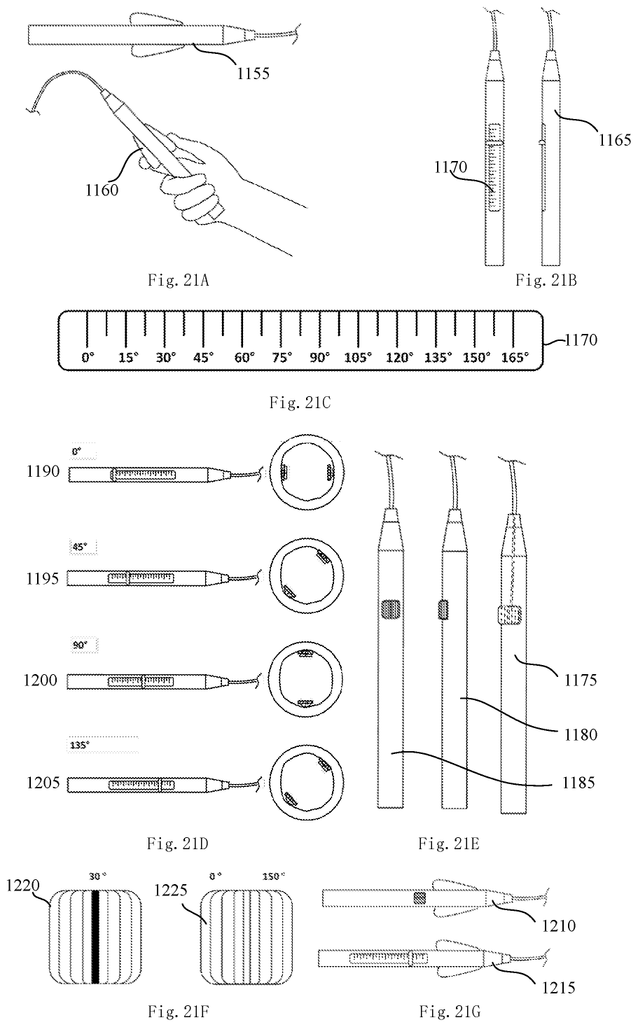

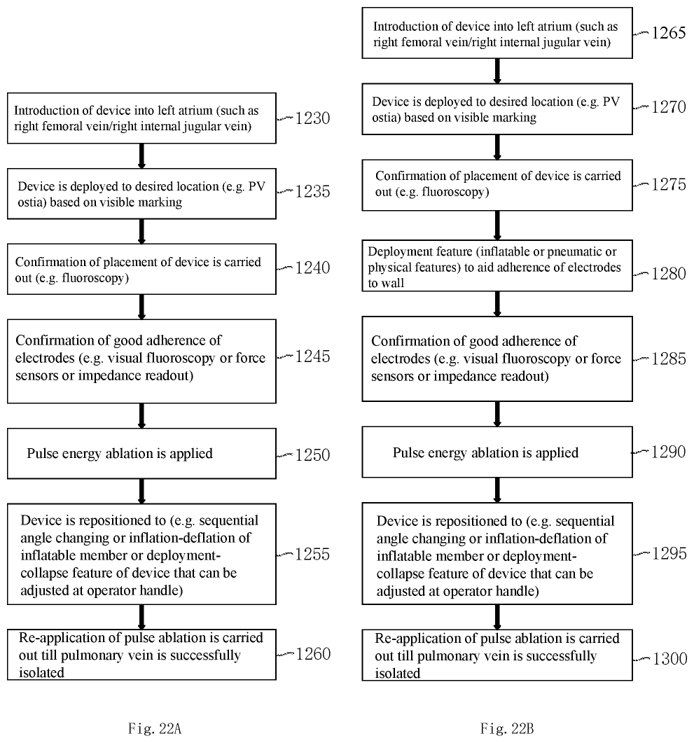

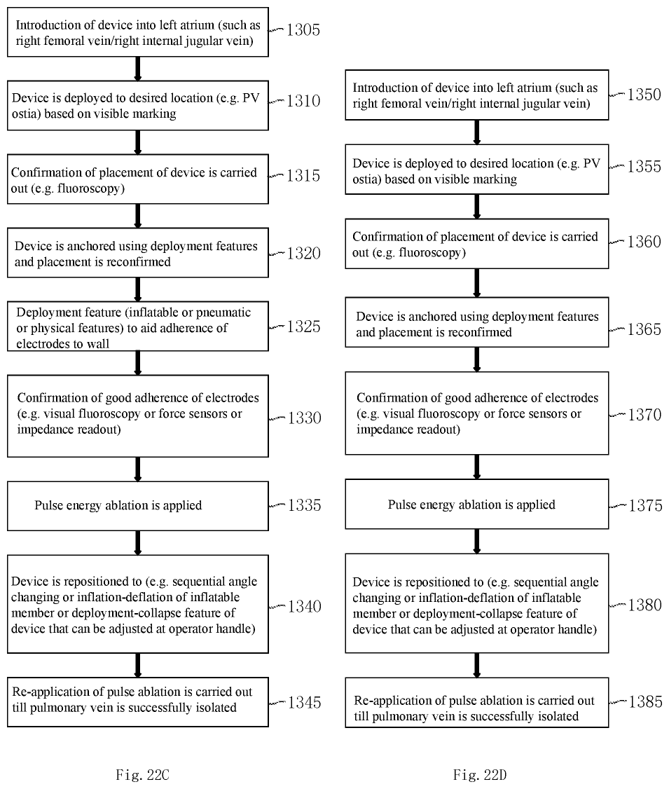

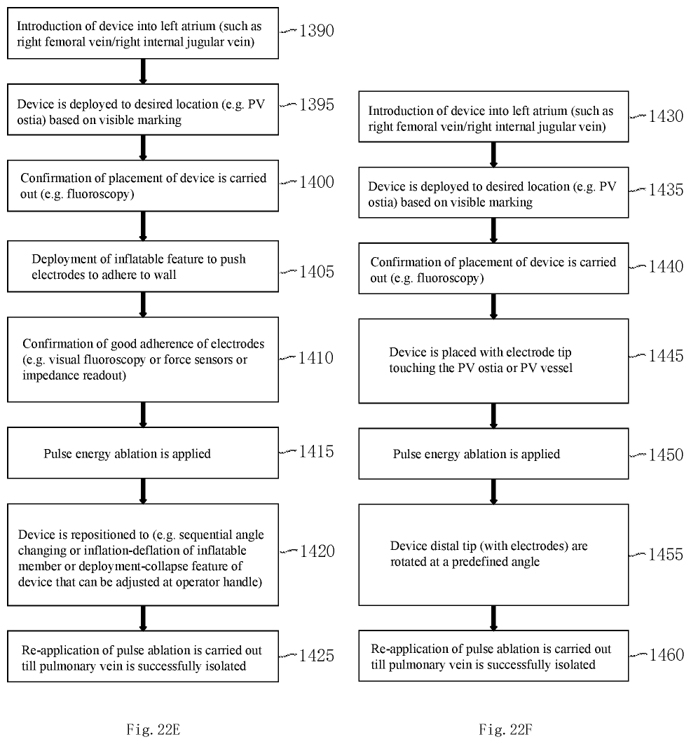

FIGS. 8A to 8G and FIGS. 9A to 9D show various embodiments similar to the flower embodiment of FIG. 5. The embodiments generally vary from FIG. 5 in that the ablation elements. While petal-like, the ablation elements in these embodiments are more elongate, resembling tentacles.

FIGS. 8A to 8B are various views according to the eighth embodiment of the present invention; FIGS. 8A and 8B show the ablation elements 426 with electrodes 432 and sensors positioned within the tip of the catheter and mounted on a pneumatic layer 430.

FIGS. 8C to 8D are various views according to the ninth embodiment of the present invention;

FIGS. 8C and 8D show the ablation elements 435 without electrode and sensors, with multiple channels existing within each ablation element 435 that are interconnected and with a rigid material at base layer 440 and multiple channels pneumatic layer on top of the ablation elements 435.

FIGS. 8E to 8F are various views according to the tenth embodiment of the present invention; FIGS. 8E and 8F show the ablation elements 445 with “mini muscles” shaped raised portion 455—raised portions having a ripple shape progressing along the contact surface—and having electrodes 450 and sensor attached to the tip of catheter and mounted on a pneumatic layer.

FIGS. 8G and 8H show the “mini muscles” shaped raised portion structure, with multiple channels within each “mini muscle” segment interconnected to other “mini muscle” segments but its surface without electrodes and sensors. The “mini muscle” segments structure include a rigid material at base layer 465 and multiple channels pneumatic layer on top.

FIGS. 8I to 8K are various views according to the twelfth embodiment of the present invention; FIGS. 8I to 8K show the various modes of pneumatic system 485, including a pneumatic layout 470 of internal multiple channels, a pneumatic layout 475 of internal multiple channels and external electrode arrangements, and a pneumatic layout 480 of external electrode arrangements.

FIGS. 9A to 9B are various views according to the thirteenth embodiment of the present invention; FIGS. 9A to 9B show an individual “tentacle” structure; FIG. 9A shows a structure 490 with a pneumatic system 495 and electrodes 500 attached to an external shell before deployment; FIG. 9B shows a structure 505 with an internal pneumatic system 510 and electrodes 500 attached to an external shell after deployment.

FIGS. 9C to 9D are various views according to the fourteenth embodiment of the present invention; FIGS. 9C to 9D show an individual “tentacle” structure; FIG. 9C shows a structure 515 with an internal pneumatic system 520 and external shell before deployment; FIG. 9D shows a structure 525 with an internal pneumatic system 530 and external shell after deployment.

FIGS. 10A to 10D are various views according to the fifteenth embodiment of the present invention; FIGS. 10A to 10D show a device 535. This embodiment uses a combination of non-compliant balloons (inflated by pressure—polyester/nylon materials as candidates) and compliant balloons (inflated by volume—polyurethane/silicone materials as candidate) as ablation element to achieve the better contact with the tissue. The arrow in FIG. 10D shows the forced direction 555 of the resilient engagement of the ablation element to the tissue.

There are several different types of inflatable array combinations, with the following three types being examples:
i) use of non-compliant balloons (inflated by pressure—polyester/nylon materials as candidates);
ii) compliant balloons (inflated by volume—polyurethane/silicone materials as candidate), and;
iii) a combination of both compliant and non-compliant balloons.

In this arrangement, the head 540 comprises a ring of elements having alternating inflatable non-compliant balloon elements 545 and compliant balloon elements 550. On inflation, the differential elements expand the ring to match the shape of the ostia, whether it be circular tissue 60 or ovoid tissue 62.

When the ablation element expands to states 560, 565, 570, the variable expansion results a combination of resilient displacement by the rigid element compliant balloon element 550 and resilient deformation by the inflatable element non-compliant balloon element 545.

FIGS. 11A to 11H are various views according to the sixteenth embodiment of the present invention; FIGS. 11A to 11H show several embodiments and additional features for use with other embodiments.

Firstly, the cardiac ablation device 575 includes a head 580 of inflatable array of ablation elements 585 with electrodes and sensors (electrodes and sensors not shown, see ablation element 605 in FIG. 11D). The ablation elements 595 can be seen before inflation shown in FIG. 11C. The ablation elements 600 can be seen after inflation shown in FIG. 11E.

Notably, FIGS. 11C and 11E also show the effect of selective inflation, where it is used to shape the head 580 depending upon the shape of the PV ostia, and so able to accommodate a circular ostia tissue 60 and an ovoid tissue 62. This selective inflation means the ablation elements can remain uninflated 620, partially inflated 630 or fully inflated 625, in order to achieve the desired shape, and corresponding resilient engagement. The resilient engagement forced directions 610, 615, 628, and 632 of the ablation element to the tissue are shown in FIGS. 11C, 11E, and 11F respectively.

Another embodiment shown in FIGS. 11D and 11G is the anchorage 590 in the form of a balloon at a distal end of the catheter and positioned to wedge the device 575 in place by selectively inflating the anchorage 590 further in the pulmonary vein, away from the ostia. In this embodiment, the balloon is ring shaped to provide an annular resilient engagement with the vein wall, but permit blood flow through the annulus. It will be appreciated, and described later, that a full balloon may also be used which may seal the vein. This may have the advantage of a greater resilient force being applied and so possibly provide a greater anchorage force. Each of these anchorage embodiments may be used with any or most of the embodiments described herein, and not limited to that shown in FIG. 11.

FIGS. 12A to 12F are various views according to the seventeenth embodiment of the present invention; The device 635 includes ablation elements 640 located at the head, some or each of which have rigid sections 650, 665, and inflatable sections 645, 655, 660, 670. As the ablation elements 640 is inflated, the rigid section prevents uniform expansion and so deforms the element.

Further, the elements may include separate chambers in each element, which may provide partially deformation of, for example, inflatable sections 645, 660, or greater deformation of, for example, inflatable sections 655, 670 as the chambers are selectively inflated, (partially or fully or not at all). As with previous embodiments, this adaptability permits the head 640 to fit circular tissue 60 or ovoid ostia tissue 62, and vary the resilient deformation. FIG. 12F shows the resilient deformation forced directions 580 and 685 in which the ablation element adapts to different tissues.

FIGS. 13A to 13F show various combinations of previously described embodiments of the anchorage balloons in use with other device embodiments; FIGS. 13A to 13B are views according to the eighteenth embodiment of the present invention; FIGS. 13C to 13D are views according to the nineteenth embodiment of the present invention; FIG. 13E are isometric views according to the twentieth embodiment of the present invention; FIG. 13F are isometric views according to the twenty-first embodiment of the present invention, including:
i) Annular anchorage balloons 695, 725, 732, which provide an bore to allow blood flow to pass through—FIGS. 13A, 13B, 13E;

The device 690 in FIG. 13A includes a balloon 700 and a balloon 705, and the device 710 in FIG. 13C includes a balloon 715 and a balloon 720. The function of the balloon is described in the seventh embodiment. The device 730 in FIGS. 13E and 13F can be described with reference to the sixth embodiment.

FIGS. 14A to 14K are various views according to the twenty second embodiment of the present invention; FIGS. 14A to 14J show a further embodiment of a device 740 having an elongate head 745. In this embodiment, the two ablation elements 755 are laterally extendable on the opposite sides of the elongated head 745, with the branches 760 acting to provide the resilient engagement, as well as providing a conduit through which the energy pulse may be delivered, and for the transfer of data if sensors are being used.

For convenient delivery, when in the retracted position shown in FIG. 14A, see FIG. 14E, the ablation elements 755 flush with the head, forming a distal cap of the elongate head 745. When in position, the ablation elements 755 extend and simultaneously open a distal aperture of the head 745. From the distal aperture, an anchorage balloon 765 is deployable from a deflated condition 775 within the head to an inflated position projecting distally 785 from the head. Once the balloon is fully deployed and the elements fully extend to the inflated position 785, ablation may commence.

This embodiment also shows the process of anchorage balloon deployment, which in this case is for a full anchorage balloon 750 but could also be used for an annular anchorage balloon.

The balloon 765 is uninflated within the head 745, in a flush state 770. As the ablation elements 755 are laterally deployed from the head 745, this opens the casing forming the head to allow the advancement of the balloon 765, in a forward state 775, followed by transition state 780 until the inflation position states 785, 790 to complete the full deployment.

Thus, in operation in the heart 795, the anchorage balloon 750 anchors the device 740 such that lateral deployment of the ablation elements 755 aligns with the ostia tissue 60 for treatment to commence.

FIG. 14K shows a further embodiment of an anchorage balloon 754. In this embodiment, the actual balloon 754 is similar to that described with respect to other embodiments. It is appreciated that maintain blood flow through the vein is highly desirable during the ablation process. As an alternative to the annular anchorage balloon 735 of FIG. 13E or other embodiments, the catheter 756 has been adapted at the distal end to allow the flow of blood through the catheter, bypassing the blockage caused by the balloon 754. Specifically, the catheter 756 at the distal end includes apertures 758 for receiving a blood flow, the blood flow passing through the catheter and exits through apertures 762, forming a bypass that allows blood to flow. In FIG. 14K, arrow 764 indicates the direction of blood flow inlet, and arrow 766 indicates the direction of blood flow outlet. Thus, the use of this bypass balloon 754 arrangement provides for limited disruption of blood flow once in place.

FIGS. 15A to 15C are various views of the twenty third embodiment of the present invention. The device 800 includes ablation elements 805 and a balloon 810. The ablation elements 805 is further embodiment of a rigid element with hinges that are pivotally deployed laterally, then released and deployed to states 815, 820, 825, 830, 835.

FIGS. 16A to 160 are views of the 24th embodiment of the present invention. FIGS. 16A to 160 show a device 840 of further embodiment, also displaying a full anchorage balloon 845. In this embodiment of lateral deployment of the elements 850, the connection to the catheter is via an expanding assembly 855. The expanding assembly 855 includes a sliding connection and a fixed connection, the sliding connection composed of an end component 860, a spline component 865, a separator 870, a spline component 875, and a fixed component 880, such that the assembly laterally deploys the ablation elements 850 in triangulating mechanism, the end component 860 to keep spline components 865 in place together with spline components 875, the spline component 865 and spline component 875 attached to electrodes 852 to enable sliding (collapse/expansion) of electrodes, the separator 870 separating 2 spline components used to support electrodes 852, the fixed component 880 keeping spline components in place together with end component 860. The expanding assembly 855 provides electrical connection for the electrodes (and sensors if required) through electrically isolating each portion of the assembly.

The elements may take several different shapes and orientations, including half circle discs 890 with adjacent polarity quarter circles, annular rings 895, half circular cylinders 900, 905, 915 as well as half circular blocks 910 having various recesses arrangements 930, 935, 940 to accommodate placement of sensors. The laterally deployed elements act to spot treat the tissue 925.

FIGS. 17A to 17J are views of the 25th embodiment of the present invention. The head 945 comprises a plurality of elongate inflatable ablation elements 950 aligned in parallel with the catheter 985. The position of the inflatable ablation elements 950 is able to shift through the resilient engagement between insertion on a circular ostia tissue 60 or ovoid tissue 62. Two different embodiments are show with the first having the catheter with a single conduit to uniformly inflate the ablation elements (FIG. 17C); And the device 975 of the second embodiment, whereby the catheter includes a plurality of conduits 985, so as to selectively inflate the elongate balloon ablation elements 980, and thus further enhance the ability to deform the head for better contact with tissue of varying shapes.

FIG. 17G shows balloon ablation elements 1005 before inflation and balloon ablation elements 1010 after inflation. FIG. 17H shows balloon ablation elements with alternate inlet structure 1015 and ablation elements with single inlet structure 1020.

FIG. 17I shows balloon ablation elements 1035 with electrodes 1040, 1045 embedded on surface of balloon and balloon ablation elements 1050 with electrodes 1060, 1055 embedded on webs. FIG. 17J shows stipulated electric field of device with electrodes that mounted on balloon ablation element surface facing PV vessel for a circular vessel tissue 60 and ovoid vessel tissue 62.

FIGS. 18A to 18D are views of the twenty sixth embodiment of the present invention. FIGS. 18A to 18D show a further embodiment of laterally deployed ablation elements, a device 1064 comprising ablation elements and a housing 1075, whereby the ablation elements are coupled to a housing 1075 of the catheter. The ablation elements are shaped similar to the jaws 1065 of a crane grab, such that on insertion the jaws 1065 are closed, and are opened to pivotally deploy the ablation elements, laterally. The jaws 1065 each have a surface arranged to face the tissue when open, with the face bearing the electrodes. The jaws 1065 may be sized so as to expand the size of the vein so as to improve contact. To this end, the degree of opening of the jaws 1065 may be selectively controllable and so a partial opening of the jaws 1065 may provide sufficient resilient engagement with the tissue to be treated.

Alternatively, the jaws may be flexible between pivots and so bend across the face for resilient deformation.

Opening the jaw 1065 may be achieved by pulling the ties 1070, so that when the ties 1070 is pulled by the action 1080, the jaw 1065 will open against resilient deformation to maintain the closing trend of the jaw 1065.

FIGS. 19A to 19F are views of the 27th embodiment of the present invention, showing a device 1085 with a head 1095 of the ablation element 1100. The ablation elements 1100 are fixed relative to the head 1100, but can extend from the head 1095 having a lever arm 1099. As shown in FIG. 19F, the head 1130 and ablation element 1135 may also be shaped to better suit an ovoid ostia tissue 62.

The adjacent elements are of opposed polarity 1105 and so on insertion, the head 1095 is progressively passed 1110, 1115, 1120 about the ostia tissue 62 in order to apply the electrical pulse and achieve the electroporation voltage delivery 1125 after sequential positioning of device.

It will be appreciated that the thickness of the head and length of the lever arm can be designed and will determine the degree of resilient deformation the element is capable of achieving.

FIG. 20A shows electroporation voltage delivery 1140 (PV cross section 62) after application of pulse energy ablation device. FIG. 20B shows the ablation energy 1142 (PV cross section 62) after application of pulse energy ablation device. FIG. 20C shows a heart indicating both an ablation pattern 1150 using traditional RF treatment and ablation pattern 1152 using pulse ablation of device according to the present invention.

FIG. 21A is a side view of deployment feature 1155 that enables a user to control electrode positioning at distal and user handling 1160 of a device according to one embodiment of the present invention with one hand.

FIG. 21B is a frontal view 1170 of deployment feature that enables user to control electrode angle within PV ostia/vessel space and side view 1165a device according to one embodiment of the present invention.

FIG. 21C shows markings 1170 on device user interface that enables user to control the angle (illustration of a 15° step angle control, the markings are as follows 0°, 15°, 30°, 45°, 60°, 75°, 90°, 105°, 120°, 135°, 150°, and 165°).

FIG. 21D shows angle markings on a device user interface enables user to control the angle 1190, 1195, 1200, 1205 and rotation of device at distal end by pushing the “button/toggle” at predetermined markings.

FIG. 21E shows frontal view of deployment 1185, side view of deployment 1180, and internal view of deployment 1175 that enables user to control electrode angle within lumen.

FIG. 21F shows angle marking in the device user interface that enables user to control the electrode angles at distal end of device by rotating the knob/dial on the handle segment of device. In this case, a 30° step angle control 1220, the markings are as follows 0°, 30°, 60°, 90°, 120°, and 150° 1225.

FIG. 21G shows designs of handle component of device, comprising of deployments 1210, 1215 to inflate balloon structure, deploy electrodes, and control the position of electrodes.

FIG. 22A is a flow diagram illustrating a method to deploy device for tissue ablation in PV ostia/vessel: for device with multiple electrodes (with or without force sensors), comprising the following steps:
1230. Introduction of device into left atrium (such as right femoral vein/right internal jugular vein);
1235. Device is deployed to desired location (e.g. PV ostia) based on visible marking;
1240. Confirmation of placement of device is carried out (e.g. fluoroscopy);
1245. Confirmation of good adherence of electrodes (e.g. visual fluoroscopy or force sensors or impedance readout);
1250. Pulse energy ablation is applied;
1255. Device is repositioned to (e.g. sequential angle changing or inflation-deflation of inflatable member or deployment-collapse feature of device that can be adjusted at operator handle);
1260. Re-application of pulse ablation is carried out till pulmonary vein is successfully isolated.

FIG. 22B is a flow diagram illustrating a method to deploy device for tissue ablation in PV ostia/vessel: for device with multiple electrodes and features to aid better electrodes alignment to vessel wall, comprising the following steps:
1265. Introduction of device into left atrium (such as right femoral vein/right internal jugular vein);
1270. Device is deployed to desired location (e.g. PV ostia) based on visible marking;
1275. Confirmation of placement of device is carried out (e.g. fluoroscopy);
1280. Deployment feature (inflatable or pneumatic or physical features) to aid adherence of electrodes to wall;
1285. Confirmation of good adherence of electrodes (e.g. visual fluoroscopy or force sensors or impedance readout);
1290. Pulse energy ablation is applied;
1295. Device is repositioned to (e.g. sequential angle changing or inflation-deflation of inflatable member or deployment-collapse feature of device that can be adjusted at operator handle);
1300. Re-application of pulse ablation is carried out till pulmonary vein is successfully isolated.

FIG. 22C is a flow diagram illustrating a method to deploy device for tissue ablation in PV ostia/vessel: for device with independent anchorage feature, multiple electrodes, features to aid better electrodes alignment to vessel wall, comprising the following steps:
1305. Introduction of device into left atrium (such as right femoral vein/right internal jugular vein);
1310. Device is deployed to desired location (e.g. PV ostia) based on visible marking;
1315. Confirmation of placement of device is carried out (e.g. fluoroscopy);
1320. Device is anchored using deployment features and placement is reconfirmed;
1325. Deployment feature (inflatable or pneumatic or physical features) to aid adherence of electrodes to wall;
1330. Confirmation of good adherence of electrodes (e.g. visual fluoroscopy or force sensors or impedance readout);
1335. Pulse energy ablation is applied;
1340. Device is repositioned to (e.g. sequential angle changing or inflation-deflation of inflatable member or deployment-collapse feature of device that can be adjusted at operator handle);
1345. Re-application of pulse ablation is carried out till pulmonary vein is successfully isolated.

FIG. 22D is a flow diagram illustrating a method to deploy device for tissue ablation in PV vessel: for device with multiple electrodes (with or without force sensors) and independent anchorage feature, comprising the following steps:
1350. Introduction of device into left atrium (such as right femoral vein/right internal jugular vein);
1355. Device is deployed to desired location (e.g. PV ostia) based on visible marking;
1360. Confirmation of placement of device is carried out (e.g. fluoroscopy);
1365. Device is anchored using deployment features and placement is reconfirmed;
1370. Confirmation of good adherence of electrodes (e.g. visual fluoroscopy or force sensors or impedance readout);
1375. Pulse energy ablation is applied;
1380. Device is repositioned to (e.g. sequential angle changing or inflation-deflation of inflatable member or deployment-collapse feature of device that can be adjusted at operator handle);
1385. Re-application of pulse ablation is carried out till pulmonary vein is successfully isolated.

FIG. 22E is a flow diagram illustrating a method to deploy device for tissue ablation in PV vessel: for device with multiple electrodes (with or without force sensors) and simultaneous anchorage feature, comprising the following steps:
1390. Introduction of device into left atrium (such as right femoral vein/right internal jugular vein);
1395. Device is deployed to desired location (e.g. PV ostia) based on visible marking;
1400. Confirmation of placement of device is carried out (e.g. fluoroscopy);
1405. Deployment of inflatable feature to push electrodes to adhere to wall;
1410. Confirmation of good adherence of electrodes (e.g. visual fluoroscopy or force sensors or impedance readout);
1415. Pulse energy ablation is applied;
1420. Device is repositioned to (e.g. sequential angle changing or inflation-deflation of inflatable member or deployment-collapse feature of device that can be adjusted at operator handle);
1425. Re-application of pulse ablation is carried out till pulmonary vein is successfully isolated.

FIG. 22F is a flow diagram illustrating a method to deploy device for tissue ablation in PV vessel: for device with electrodes designed to achieve good adherence to vessel wall when an operator manoeuvres it, comprising the following steps:
1430. Introduction of device into left atrium (such as right femoral vein/right internal jugular vein);
1435. Device is deployed to desired location (e.g. PV ostia) based on visible marking;
1440. Confirmation of placement of device is carried out (e.g. fluoroscopy);
1445. Device is placed with electrode tip touching the PV ostia or PV vessel;
1450. Pulse energy ablation is applied;
1455. Device distal tip (with electrodes) are rotated at a predefined angle;
1460. Re-application of pulse ablation is carried out till pulmonary vein is successfully isolated.

The above embodiments are only intended to illustrate the technical concept and characteristics of the invention and enable those familiar with the technology to understand the content of the invention and implement it accordingly, not limiting the scope of protection of the invention. Any equivalent changes or modifications made in accordance with the spirit of the invention should be covered by the scope of protection of the invention.

1. A cardiac ablation system for treating cardiac tissue comprises a catheter and a head, the head is the ablation end of the cardiac ablation system for cardiac tissue, and the catheter is a flexible tubular connector used by the cardiac ablation system to couple to the head, wherein:
the head has a plurality of individual ablation elements; each ablation element is provided with a contact surface for contacting cardiac tissue and a flexible support body for supporting the contact surface, wherein an energy acting part is arranged on the contact surface;
the ablation element has two operating states of contraction and extension at the head position of the cardiac ablation system; in the contraction state, a plurality of individual ablation elements aggregate with each other and present a minimum volume; in the extension state, one or more mutually individual ablation elements open and adapt to various shape changes at the point where the cardiac tissue is contacted through the contact surfaces on each ablation element.

2. The system according to claim 1, wherein at least some of the ablation elements are arranged for resilient engagement with the tissue.

3. The system according to claim 2, wherein all the ablation elements are arranged for resilient engagement with the tissue.

4. The system according to claim 2, wherein the resilient engagement comprises one or both of resilient deformation and resilient displacement.

5. The system according to claim 4, wherein the resilient deformation of the ablation element includes inflation of the ablation element.

6. The system according to claim 5, wherein the ablation elements are selectively inflatable.

7. The system according to claim 6, wherein the selective inflation includes varying inflation of the ablation elements, such that the head includes ablation elements of different inflation.

8. The system according to claim 5, wherein each ablation element includes a rigid section, such that on inflation the ablation element is arranged to deform about the rigid section through differential expansion.

9. The system according to claim 5, wherein each head includes at least one compliant balloon element and at least one non-compliant balloon element, the non-compliant balloon and the compliant balloon elements forming a ring arranged to contact the tissue.

10. The system according to claim 1, wherein the head comprises a plurality of the elongate ablation elements.

11. The system according to claim 10, wherein the elongate ablation elements are individually movable and substantially rigid, the resilient engagement comprising resilient displacement.

12. The system according to claim 10, wherein the head includes a selectively movable housing, the housing arranged to selectively move the ablation elements from a distal or proximal position to a position opposite the distal or proximal position, such that the elongate ablation elements are arranged to resiliently project radially from a centre of the head.

13. The system according to claim 10, wherein the head includes at least one inflatable balloon, the balloon coupled to the head and the ablation elements are arranged around a peripheral edge of the balloon, wherein on inflation the balloon is arranged to bias the elongate ablation elements to displaced so as project radially outwards from a centre of the head.

14. The system according to claim 13, wherein there are a plurality of balloons coupled to the head, the balloons selectively inflatable, the balloons arranged to be of a different inflation, the elements arranged to be displaced differently

15. The system according to claim 13, wherein the balloons are annular.

16. The system according to claim 1, wherein the head comprises a pair of elongate linkage ablation elements, the ablation elements comprising a linear arrangement of segments pivotally connected, and arranged to project radially from a centre of the head.

17. The system according to claim 16, wherein the ablation elements are each connected at the end to the head and arranged to move from a position parallel to the catheter to the radially projected position on application of a tensile force applied to the end.

18. The system according to claim 1, wherein the head comprises a plurality of curved ablation elements, the ablation elements are curved and arranged to project radially from a centre of the head, said resilient engagement including resilient deformation of the ablation elements.

19. The system according to claim 18, further including a pneumatic portion of the ablation element, the contact surface located on the pneumatic portion.

20. The system according to claim 18, wherein the contact surface is profiled.

21. The system according to claim 1, wherein the ablation elements are arranged to move from a retracted position flush with the head to a laterally extended position, the ablation elements attached to the head via branches

22. The system according to claim 21, further including an anchorage balloon, the anchorage balloon arranged to move from a deflated position within the head to a deployed position extending in a distal direction from the head.

23. The system according to claim 1, further including an anchorage for fixing the head relative to the tissue.

24. The system according to claim 23, wherein the anchorage includes an inflatable anchorage balloon coupled to the catheter, the inflatable anchorage balloon arranged to engage walls of a vein adjacent to the tissue.

25. The system according to claim 24, wherein the anchorage balloon is annular, having a bore arranged to allow the passing of blood flow.
